(19) United States
(12) Patent Application Publication
(10) Pub. No.: US 2008/0233582 A1
(43) Pub. Date: Sep. 25, 2008

(54) SINGLE NUCLEOTIDE POLYMORPHISMS ASSOCIATED WITH SUSCEPTIBILITY TO CARDIOVASCULAR DISEASE

(75) Inventors: Monika Stoll, Muenster (DE); Gerd Assmann, Munster (DE); Helmut Schulte, Munster (DE); Werner Kroll, Wayland, MA (US); Stephan Schwers, Koln (DE); Christoph Petry, Monchengladbach (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Healthcare Diagnostics Inc., Tarrytown, NY (US)

(21) Appl. No.: 12/019,651

(22) Filed: Jan. 25, 2008

Related U.S. Application Data
(63) Continuation-in-part of application No. PCT/US2006/029449, filed on Jul. 26, 2006.
(60) Provisional application No. 60/702,760, filed on Jul. 26, 2005.
(60) Provisional application No. 60/703,219, filed on Jul. 27, 2005.
(60) Provisional application No. 60/708,719, filed on Aug. 15, 2005.
(60) Provisional application No. 60/742,407, filed on Dec. 5, 2005.

(30) Foreign Application Priority Data
Jun. 21, 2006 (EP) 06012722.2
Apr. 18, 2007 (EP) 07251633

(51) Int. Cl. C12Q 1/68; C07H 21/00

(52) U.S. Cl. 435/6; 536/24.31

(57) ABSTRACT

The present invention provides SNPs, polymorphic variants, and haplotypes associated with cardiovascular disease. The invention also provides methods for detecting the SNPs, polymorphic variants, and haplotypes. The invention also provides methods for determining an individual's genotype with respect to one or more polymorphisms and/or haplotypes associated with cardiovascular disease. The invention further provides methods of determining whether an individual has or is susceptible to development or occurrence of a cardiovascular disease or event. The methods are useful for providing diagnostic and/or prognostic information, selecting therapeutic regimens, etc. The invention further provides reagents and kits for practicing the methods.

### CROSS REFERENCE TO RELATED APPLICATIONS

The present application is a continuation-in-part application of International Application No. PCT/US2006/029449, filed Jul. 6, 2006. The present application is co-pending with, shares at least one common inventor with, and hereby claims priority to U.S. Provisional Patent Application Ser. No. 60/702,760, filed Jul. 26, 2005. The present application is also co-pending with, shares at least one common inventor with, and hereby claims priority to U.S. Provisional Patent Application Ser. No. 60/703,219, filed Jul. 27, 2005. The present application is co-pending with, shares at least one common inventor with, and hereby claims priority to U.S. Provisional Patent Application Ser. No. 60/708,719, filed Aug. 15, 2005. The present application is co-pending with, shares at least one common inventor with, and hereby claims priority to U.S. Provisional Patent Application Ser. No. 60/742,407, filed Dec. 5, 2005. The present application is co-pending with, shares at least one common inventor with, and hereby claims priority to European Patent Application Serial Number 06012722.2, filed Jun. 21, 2006 and European Patent Application Serial Number 07251633, filed Apr. 18, 2007. Each of these applications is incorporated herein by reference in its entirety.

### BACKGROUND OF THE INVENTION

Cardiovascular diseases and conditions are a major cause of morbidity and mortality throughout the world. These diseases and conditions include, but are not limited to, the various disorders of the heart and the vascular system typically referred to as myocardial infarction (heart attack), atherosclerosis, ischemic heart disease, coronary artery disease, congestive heart failure, atrial and ventricular arrhythmias, hypertensive vascular diseases, and peripheral vascular diseases.

Atherosclerosis is a principal causative agent of heart attack and stroke. Atherosclerosis is a complex disease involving many cell types and molecular factors (for a detailed review, see Ross, Nature 362: 801-809, 1993; and Lusis, A. J., Nature 407, 233-241, 2000). The process involves the formation of fibrofatty and fibrous lesions or plaques in the vessel wall, preceded and accompanied by inflammation. Such plaques can partially or fully occlude the blood vessel concerned and thus restrict the flow of blood, resulting in ischemia. Ischemia is a general term that refers to a condition characterized by inadequate blood flow to an area of the body such as an organ or tissue, resulting in an insufficient oxygen supply. Ischemia is most frequently caused by a narrowing or complete obstruction of the arteries and may occur in any organ or tissue. The most common cause of ischemia in the heart is atherosclerotic disease of the coronary arteries, also referred to as coronary heart disease or coronary artery disease. By reducing the lumen of these vessels, atherosclerosis causes an absolute decrease in myocardial perfusion in the basal state and/or limits appropriate increases in perfusion when the demand for flow is augmented. Coronary blood flow can also be limited by arterial thrombi, spasm, and, rarely, coronary emboli, as well as by ostial narrowing due to luetic aortitis. Myocardial ischemia can also occur if myocardial oxygen demands are abnormally increased, as in severe ventricular hypertrophy due to hypertension or aortic stenosis. Severe and prolonged myocardial ischemia can lead to myocardial infarction. The triggering event for a myocardial infarction is often the rupture of an atherosclerotic plaque, leading to a blood clot that causes a sudden decrease in vessel lumen.

Approximately half of all first myocardial infarctions are fatal. Furthermore, in many instances coronary heart disease develops silently, and there may be no warning symptoms, such as chest pain, prior to onset of the heart attack. The development of effective strategies to prevent coronary artery disease and to inhibit its progression is therefore of considerable importance.

A number of approaches are currently employed for the treatment and/or prevention of cardiovascular diseases, e.g., atherosclerosis and coronary artery disease. Pharmaceutically based therapies include lipid lowering agents (e.g., statins), aspirin and other anti-platelet agents, and anti-hypertensive medications. Lifestyle modification also plays an important role since it is known that factors such as smoking, obesity, and a high fat diet increase the risk of myocardial infarction.

It is thought that genetic factors contribute to the development of atherosclerosis and coronary artery disease (Scheuner, M T, Genet Med. July August;5(4):269-85, 2003). An individual's genetic makeup is therefore a significant determinant of the likelihood that he or she will suffer a myocardial infarction, particularly at a young age. However, while a “family history” of heart disease is a significant risk factor, many heart attack victims lack such a family history and, conversely, not all individuals with such a family history do indeed develop the disease. Thus the nature of the genetic contribution to cardiovascular disease is unclear.

There is a need in the art for methods and accompanying reagents that can be used to better assess an individual's susceptibility of developing cardiovascular disease. The need for such methods and reagents is especially acute in view of the fact that, atherosclerosis frequently remains clinically silent in its early stages and yet is often evident at post-mortem examination even among individuals in their teens and twenties (McGill, H. C. Jr & McMahan, C. A., Am. J. Cardiol., 82, 30T-36T, 1998).

### SUMMARY OF THE INVENTION

The present invention is based at least in part on the identification of single nucleotide polymorphisms (SNPs) that are informative with respect to cardiovascular disease. In particular, the invention is based in part on the discovery that particular polymorphic variants of the polymorphic sites at which these SNPs are located are associated with an increased risk of cardiovascular disease, e.g., acute coronary events such as myocardial infarction. In certain aspects, the invention provides methods for genotyping an individual comprising steps of: providing a sample obtained from an individual in need of testing for presence of or susceptibility to a cardiovascular disease and detecting a polymorphic variant of a polymorphism listed in the column entitled “dbSNP_RS_ID” in Table 1 and/or Table 2.

Table 1 is included in International Application No. PCT/US2006/029449, U.S. Provisional Patent Application Ser. Nos. 60/702,760, 60/703,219, 60/708,719, 60/742,407, and European Patent Application Serial Number 06012722.2 and its contents are part of this specification and is incorporated herein by reference in its entirety. Table 2 is included in European Application No. 07251633 and its contents are part of this specification and is incorporated herein by reference in its entirety. The contents of Tables 1 and 2 are described in detail below.

In a certain embodiments, the invention provides methods for testing the presence of a polymorphism in the sequences of a gene listed in Table 1 and/or Table 2. In certain embodiments, the invention provides methods for demonstrating a link between particular allelic variants of the polymorphisms listed in Tables 1 and 2 and a susceptibility to cardiovascular disease by showing that allele frequencies at the polymorphic sites differ significantly among individuals who suffered a myocardial infarction at an early age (<50 years of age) as compared to individuals who did not suffer a myocardial infarction but had a similar pattern of classical risk factors. An individual having a particular allelic variant or combination of allelic variants, e.g., having a particular genotype with respect to one or more allelic variants is considered to have “susceptibility to cardiovascular disease” if (i) the individual is more likely to develop a cardiovascular disease or manifest a symptom or sign of cardiovascular disease than a comparable individual having a different genotype with respect to those allelic variant(s), wherein the comparable individual is otherwise similar with respect to one or more (e.g., all) of the classical CVD risk factors, and/or (ii) the individual is more likely to develop a cardiovascular disease or manifest a symptom or sign of cardiovascular disease than an individual of a similar age (e.g., up to 5 years older or younger) and the same sex but having a different genotype with respect to those allelic variant(s). It will be appreciated that various cardiovascular diseases are interrelated, and the existence of a particular cardiovascular disease or condition may contribute to the development of other(s). For example, an individual who has suffered a myocardial infarction (“MI”) may have an increased risk of having a cardiac arrhythmia and may have an increased risk of heart failure.

In certain aspects, the invention provides methods of diagnosing cardiovascular disease or susceptibility to development of cardiovascular disease in an individual, said method comprising determining one, more than one, or all genotypes in said individual of the polymorphisms listed in Table 1 and/or Table 2.

In certain aspects, the invention provides a variety of reagents and kits for use in detecting a polymorphic variant of a polymorphism listed in Table 1 and/or Table 2.

The invention encompasses the recognition that the ability to classify individuals who are at increased risk of myocardial infarction on the basis of their genotype allows the establishment of a correlation between genotype and response to particular therapeutic regimens.

The invention also encompasses the recognition that an integrated assessment of an individual's risk, and/or an integrated assessment of the appropriate therapeutic regimen for a particular individual, can include an assessment of both genetic and of non-genetic factors, which can be combined in a variety of different ways. The availability of methods and reagents for evaluating the genetic factors associated with cardiovascular disease allows a more accurate assessment of whether an individual would benefit from various therapeutic interventions such as administration of particular pharmaceutical agents and/or encouragement of particular lifestyle modifications.

This application refers to various patents, patent applications, journal articles, and other publications, all of which are incorporated herein by reference. In addition, the following standard reference works are incorporated herein by reference: Ausubel, F., (ed.), Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Harlow, E., et al., Antibodies: A Laboratory Manual, (Cold Spring Harbor Laboratory Press, 2nd ed. 1988); Hardman, J., Limbird. E., Gilman, A. (Eds.), Braunwald, E., Zipes, D. P., and Libby, P. (eds.) Heart Disease: A Textbook of Cardiovascular Medicine. W B Saunders; 6th edition (Feb. 15, 2001); Chien, K. R., Molecular Basis of Cardiovascular Disease: A Companion to Braunwald's Heart Disease, W B Saunders; Revised edition (2003); and Goodman and Gilman's The Pharmacological Basis of Therapeutics, 10^{th} Ed. McGraw Hill, 2001 (referred to herein as Goodman and Gilman). In the event of a conflict or inconsistency between any of the incorporated references and the instant specification or the understanding of one or ordinary skill in the art, the specification shall control, it being understood that the determination of whether a conflict or inconsistency exists is within the discretion of the inventors and can be made at any time.

### BRIEF DESCRIPTION OF THE DRAWING

FIG. 1 presents characteristic group statistics of individuals (study subjects) that were studied to identify SNPs and polymorphic variants associated with cardiovascular disease classified according to the number of subjects in different PROCAM risk categories. The column labeled “PROCAM” refers to controls, and the column entitled “MI-Patients” refers to cases. Mean and standard deviation values of PROCAM and MI-patients are shown in the right two columns.

FIG. 2 presents characteristics of individuals (study subjects) that were studied to identify SNPs and polymorphic variants associated with cardiovascular disease. The column labeled “PROCAM” refers to controls, and the column entitled “MI-Patients” refers to cases. The average values for various PROCAM risk factors and the overall average PROCAM score are provided. The percentage of each group exhibiting the particular risk factor is shown.

FIG. 3 presents risk profiles of individuals (study subjects) that were studied to identify SNPs and polymorphic variants associated with cardiovascular disease. The column labeled “PROCAM” refers to controls, and the column entitled “MI-Patients” refers to cases.

### DEFINITIONS

For purposes of convenience, definitions of a variety of terms used herein are presented below.

The term “allele” which is used interchangeably herein with the term “allelic variant” refers to alternative forms of a gene or a portion thereof. Alleles occupy the same locus or position on homologous chromosomes. When an individual has two identical alleles of a gene, the individual is said to be homozygous for the gene or allele. When an individual has two different alleles of a gene, the individual is said to be heterozygous for the gene. Alleles of a specific gene can differ from each other in a single nucleotide or in a plurality nucleotides, and can include substitutions, deletions, and/or insertions of nucleotides with respect to each other. An allele of a gene can also be a form of a gene containing a mutation.

While the terms “allele” and “allelic variant” have traditionally been applied in the context of genes, which can include a plurality of polymorphic sites, the term is also used herein to apply to any form of a genomic DNA sequence, which can be as small as a single nucleotide and may or may not fall within a gene. Thus each polymorphic variant of a polymorphic site is considered an allele, and when referring to single nucleotide polymorphisms, the terms “polymorphic variant” and “allele” are used interchangeably herein. An “allele frequency” refers to the frequency at which a particular polymorphic variant, or allele, occurs in a population being tested, e.g., in cases or controls in an association study.

The term “allelic variant of a polymorphic region of a gene” refers to a region of a gene having one of several nucleotide sequences found in that region of the gene in different individuals in a population.

“Antibody”, as used herein, refers to an immunoglobulin that binds to an antigen. An antibody may be natural or wholly or partially synthetically produced. An antibody may be derived from natural sources, e.g., purified from an animal such as a rodent, rabbit, or chicken, that has been immunized with an antigen or a construct that encodes the antigen. An antibody may be a member of any immunoglobulin class, including any of the human classes: IgG, IgM, IgA, IgD, and IgE. The antibody may be an antibody fragment such as an Fab′, F(ab′)₂, scFv (single-chain variable) or other fragment that retains an antigen binding site, or a recombinantly produced scFv fragment, including recombinantly produced fragments that comprise an immunoglobulin antigen binding domain. See, e.g., Allen, T., Nature Reviews Cancer, Vol. 2, 750-765, 2002, and references therein. Antibody fragments which contain the antigen binding site of the antibody molecule can be generated by known techniques. For example, F(ab′)₂ fragments can be produced by pepsin digestion of the antibody molecule, Fab′ fragment by reducing the disulfide bridges of the F(ab′)₂ fragment, or by treating the antibody molecule with papain and a reducing agent.

Antibodies, antibody fragments, and/or protein domains comprising an antigen binding site may be generated and/or selected in vitro, e.g., using techniques such as phage display (Winter, G. et al., Annu. Rev. Immunol. 12:433-455, 1994,1994), ribosome display (Hanes, J., and Pluckthun, A. Proc. Natl. Acad. Sci. USA. 94:4937-4942, 1997), etc. An antibody may be polyclonal (e.g., an affinity-purified polyclonal antibody) or monoclonal.

An antibody may be a “chimeric” antibody in which for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. Such an antibody is considered at least partially “humanized”. The degree to which an antibody is “humanized” can vary. Thus part or most of the variable domain of a rodent antibody may be replaced by human sequences. For example, according to one approach murine complementarity-determining regions (CDRs) are grafted onto the variable light (VL) and variable heavy (VH) frameworks of human immunoglobulin molecules, while retaining only those murine framework residues deemed essential for the integrity of the antigen-binding site. See Gonzales N R, Tumour Biol. January-February;26(1):31-43, 2005 for a review of various methods of minimizing antigenicity of a monoclonal antibody. Such human or humanized chimeric antibodies are often advantageous for use in therapy of human diseases or disorders, since the human or humanized antibodies are much less likely than to induce an immune response.

The terms “approximately” or “about” in reference to a number are generally include numbers that fall within a range of 5% in either direction (greater than or less than) of the number unless otherwise stated or otherwise evident from the context (except where such number would exceed 100% of a possible value).

“Classical CVD risk factors” as used herein, refer to 5 continuous variables (age, LDL cholesterol, HDL cholesterol, triglyceride (TG) level, and systolic blood pressure) and 3 discrete variables (smoking status, diabetes, and MI in family history), as described in Assmann, G., et al., Circulation, 105:310, 2002. In general, higher values for age, LDL, TG, systolic blood pressure), correlate with an increased risk of developing CVD and/or experiencing a major coronary event, while lower value for HDL correlates with a decreased risk. Smoking, diabetes, and MI in family history each correlates with an increased risk of developing CVD and/or experiencing a major coronary event. These risk factors were used to develop the PROCAM scoring system, which predicts the likelihood that an individual will experience a major coronary event within a defined period of time, e.g., within 10 years. One of ordinary skill in the art will recognize that alternative risk factors and scoring systems could be developed. One of ordinary skill in the art will also recognize that approximations and substitutions may be made to the aforesaid classical risk factors. For example, in some embodiments total cholesterol level could be used. The risk factors may also be augmented, e.g., to include measurements of diastolic blood pressure, measurements of blood level of C reactive protein, the effect of any particular therapeutic regimen the individual is following, and various cardiovascular status markers.

The term “complementary” is used herein in accordance with its art-accepted meaning to refer to the capacity for precise pairing between particular bases, nucleosides, nucleotides or nucleic acids. For example, adenine (A) and uracil (U) are complementary; adenine (A) and thymine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence, the nucleotides form a complementary base pair, and the nucleic acids are complementary at that position. A percent complementarity of two nucleic acids within a window of evaluation may be evaluated by determining the total number of nucleotides in both strands that form complementary base pairs within the window, dividing by the total number of nucleotides within the window, and multiplying by 100. The two nucleic acids are aligned in anti-parallel orientation for maximum complementarity over the window, allowing introduction of gaps. When computing the number of complementary nucleotides needed to achieve a particular % complementarity, fractions are rounded to the nearest whole number. A position occupied by non-complementary nucleotides constitutes a mismatch. A nucleic acid that is 100% complementary to another nucleic acid is said to be its “complement”. Thus a nucleic acid that is 100% complementary to its complement will base pair without a single mismatch. It is to be understood that where the invention provides a nucleic acid, the complement of the nucleic acid is also provided.

As used herein, “diagnostic information” is any information that is useful in determining whether a patient has or is susceptible to developing a disease or condition and/or in classifying the disease or condition into a phenotypic category or any category having significance with regards to the prognosis or severity of the disease or condition, or likely response to treatment (either treatment in general or any particular treatment) of the disease or condition. Diagnostic information can include, e.g., an assessment of the likelihood that an individual will develop a cardiovascular disease and/or will suffer a major coronary event within a defined time period, e.g., 10 years. “Diagnosis” refers to providing any type of diagnostic information, including, but not limited to, whether a subject has or is likely to have a condition (such as a cardiovascular disease), information related to the nature or classification of a disease, information related to prognosis and/or information useful in selecting an appropriate therapeutic regimen.

The term “gene”, as used herein, has its meaning as understood in the art. In most cases, a gene as used herein comprises a nucleic acid sequence that encodes a polypeptide and can also include regulatory sequences (e.g., promoters, enhancers, etc.) and/or intron sequences. It will be appreciated that a “gene” can also refer to a nucleic acid that does not encode a protein but rather encodes a functional RNA molecule such as an rRNA, tRNA, etc.

A “gene product” or “expression product” is an RNA transcribed from the gene (e.g., either pre- or post-processing) or a polypeptide encoded by an RNA transcribed from the gene (e.g., either pre- or post-modification). RNA transcribed from a gene or polynucleotide is said to be encoded by the gene or polynucleotide.

“Genotype” refers to the identity of one or more allelic variants at one or more particular polymorphic positions in an individual. It will be appreciated that an individual's genome will contain two allelic variants for each polymorphic position (located on homologous chromosomes). The allelic variants can be the same or different. A genotype can include the identity of either or both of the allelic variants. A genotype can include the identities of allelic variants at multiple different polymorphic positions, which may or may not be located within a single gene. A genotype can also refer to the identity of an allele of a gene at a particular gene locus in an individual and can include the identity of either or both alleles. The identity of the allele of a gene may include the identity of the polymorphic variants that exist at multiple polymorphic sites within the gene. The identity of an allelic variant or an allele of a gene refers to the sequence of the allelic variant or allele of a gene, e.g., the identity of the nucleotide present at a polymorphic position or the identities of nucleotides present at each of the polymorphic positions in a gene. It will be appreciated that the identity need not be provided in terms of the sequence itself For example, it is typical to assign identifiers such as +, −, A, a, B, b, etc., to different allelic variants or alleles for descriptive purposes. Any suitable identifier can be used. “Genotyping” an individual refers to providing the genotype of the individual with respect to one or more allelic variants or alleles.

“Haplotype” refers to the particular combinations of polymorphic variants (alleles) observed in a population at polymorphic sites on a single chromosome or within a region of a single chromosome. The polymorphic variants that constitute a haplotype are in linkage disequilibrium and thus tend to be inherited together.

A “haplotype block” is a region of the genome over which there is little evidence for historical recombination and within which only a few common haplotypes are observed. Haplotype blocks can vary in size and are separated by sites at which recombination can be inferred. Haplotype blocks are described e.g., in Gabriel, S. B., et al., Science, 296:2225-2229, 2002; Daly, M. J., et al., Nature Genetics, 29: 229-232, 2001; Reich, D. E., et al., Nature, 411: 199-204.

“Identity” refers to the extent to which the sequence of two or more nucleic acids is the same. The percent identity between first and second nucleic acids over a window of evaluation may be computed by aligning the nucleic acids, determining the number of nucleotides within the window of evaluation that are opposite an identical nucleotide allowing the introduction of gaps to maximize identity, dividing by the total number of nucleotides in the window, and multiplying by 100. When computing the number of identical nucleotides needed to achieve a particular % identity, fractions are to be rounded to the nearest whole number. When two or more sequences are compared, any of them may be considered the reference sequence.

Percent identity can be calculated using a variety of computer programs known in the art. For example, computer programs such as BLASTN, BLASTP, Gapped BLAST, etc., generate alignments and provide % identity between a sequence of interest and sequences in any of a variety of public databases. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877,1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, default parameters of the respective programs may be used. Alternatively, the practitioner may use non-default parameters depending on his or her experimental and/or other requirements. See the Web site having URL www.ncbi.nlm.nih.gov. A PAM250 or BLOSUM62 matrix may be used.

“Individual” means any human being.

The term “isolated” means 1) separated from at least some of the components with which it is usually associated in nature; 2) prepared or purified by a process that involves the hand of man; and/or 3) not occurring in nature. An isolated nucleic acid, such as DNA or RNA, is typically separated from other DNAs or RNAs, respectively, that are present in the natural or original source of the macromolecule. An isolated polypeptide is typically separated from other polypeptides, respectively, that are present in the natural or original source of the macromolecule, and the term is intended to encompass purified, recombinant, and synthetically produced polypeptides. The term isolated as used herein also refers to a nucleic acid or polypeptide that is substantially free of cellular material, viral material, or culture medium when produced by recombinant DNA techniques, or substantially free of chemical precursors or other chemicals when chemically synthesized. A DNA that is removed from its chromosomal location or amplified from its chromosomal location is considered “isolated”. A cDNA is also considered an isolated nucleic acid in certain embodiments. In some embodiments a nucleic acid or polypeptide is considered isolated when it is expressed in a host cell system using recombinant DNA techniques. Moreover, an “isolated nucleic acid” is meant to include nucleic acid fragments which are not naturally occurring as fragments and would not be found in the natural state. Any of the nucleic acids and polypeptides disclosed herein may be provided in isolated form.

As used herein, “linkage” or “linked” generally refers to genetic linkage. Two loci (e.g., two SNPs, a DNA marker locus and a disease locus such as a mutation causing disease, etc.) are said to be genetically linked when the probability of a recombination event occurring between these two loci is below 50% (which equals the probability of recombination between two unlinked loci). Two loci are closely linked genetically if the recombination frequency in the region between the loci is low, but may be essentially genetically unlinked or only weakly linked if the recombination frequency between the two loci is high even if they are in close physical proximity to one another along a chromosome.

“Linkage disequilibrium” or “LD” refers to a situation in which two or more allelic variants are linked, i.e., there is a non-random correlation between allelic variants at two or more polymorphic sites in individuals in a population. Two or more allelic variants that are linked are said to be in linkage disequilibrium. In general, allelic variants that are part of a haplotype or haplotype block are in linkage disequilibrium. A variety of metrics are known in the art to evaluate the extent to which any two polymorphic variants (alleles) are in LD. Suitable metrics include D′, r², and others (see, e.g., Hedrick, P. W., Genetics, 117(2):331-41, 1987). As used herein, polymorphic variants are in “strong LD” if D′>0.8.

The term “locus” refers to a position in a chromosome. For example, a locus of a gene refers to the chromosomal position of the gene.

The term “microparticle” is used herein to refer to particles having a smallest cross-sectional dimension of 50 microns or less. In certain embodiments, the smallest cross-sectional dimension of the microparticle is 10 microns or less. In certain embodiments the smallest cross-sectional dimension is approximately 3 microns or less, approximately 1 micron or less, or approximately 0.5 microns or less, e.g., approximately 0.1, 0.2, 0.3, or 0.4 microns. Microparticles may be made of a variety of inorganic or organic materials including, but not limited to, glass (e.g., controlled pore glass), silica, zirconia, cross-linked polystyrene, polyacrylate, polymethylmethacrylate, titanium dioxide, latex, polystyrene, etc. See, e.g., U.S. Pat. No. 6,406,848, for various suitable materials and other considerations. Dyna beads, available from Dynal, Oslo, Norway, are an example of commercially available microparticles of use in the present invention. Magnetically responsive microparticles can be used. In certain embodiments, one or more populations of fluorescent microparticles are employed. The populations may have different fluorescence characteristics so that they can be distinguished from one another, e.g., using flow cytometry. In some embodiments the microparticles are modified, e.g., an oligonucleotide is attached to a microparticle to serve as a “zip code” that allows specific hybridization to a second oligonucleotide that comprises a portion that is complementary to the zip code.

The term “modulation”, “modulate”, and like terms, as used herein refers to both up-regulation, (e.g., activation or stimulation), for example by agonizing, and down-regulation (e.g., inhibition or suppression), for example by antagonizing of a bioactivity (e.g. expression of a gene). Thus a “modulator” may be an agent that enhances or increases the expression and/or activity of a gene, nucleic acid, or polypeptide, or an agent that reduces or inhibits the expression and/or activity of a gene, nucleic acid, or polypeptide.

The terms “nucleic acid”, “polynucleotide”, and “oligonucleotide” are used interchangeably herein to refer to a polymer of at least three nucleotides such as deoxyribonucleic acid (DNA) and ribonucleic acid (RNA). An oligonucleotide is typically less than 100 nucleotides in length, although oligonucleotides of the present invention are not limited to such a length. A nucleotide comprises a nitrogenous base, a sugar molecule, and a phosphate group. A nucleoside comprises a nitrogenous base linked to a sugar molecule. In a polynucleotide phosphate groups covalently link adjacent nucleosides to form a polymer. The polymer may include natural nucleosides (e.g., adenosine, thymidine, guanosine, cytidine, uridine, deoxyadenosine, deoxythymidine, deoxyguanosine, and deoxycytidine), nucleoside analogs, chemically modified bases, biologically modified bases (e.g., methylated bases), intercalated bases, modified sugars (e.g., modified purines or pyrimidines). The phosphate groups in nucleic acid are typically considered to form the internucleoside backbone of the polymer. In naturally occurring nucleic acids (DNA or RNA), the backbone linkage is via a phosphodiester bond. However, polynucleotides containing modified backbones or non-naturally occurring internucleoside linkages can also be used in the present invention. See Kornberg and Baker, DNA Replication, 2nd Ed. (Freeman, San Francisco, 1992), Scheit, Nucleotide Analogs (John Wiley, New York, 1980), and U.S. Patent Publication No. 20040092470 and references therein for further discussion of various nucleotides, nucleosides, and backbone structures that can be used in the polynucleotides described herein, and methods for producing them. Nucleic acids can be single or double-stranded. Certain polynucleotides of the invention may be modified by chemical or biological means. In certain embodiments, these modifications lead to increased stability of the polynucleotide. Modifications include methylation, phosphorylation, end-capping, etc. The term “nucleic acid sequence” as used herein refers to the nucleic acid material itself and is not restricted to the sequence information (i.e. the succession of letters chosen among the five base letters A, G, C, T, or U) that biochemically characterizes a specific nucleic acid, e.g., a DNA or RNA molecule. A nucleic acid sequence is presented in the 5′ to 3′ direction unless otherwise indicated.

“Operably linked” or “operably associated” refers to a functional relationship between two nucleic acids, wherein the expression, activity, localization, etc., of one of the sequences is controlled by, directed by, regulated by, modulated by, etc., the other nucleic acid. The two nucleic acids are said to be operably linked or operably associated. “Operably linked” or “operably associated” also refers to a relationship between two polypeptides wherein the expression of one of the polypeptides is controlled by, directed by, regulated by, modulated by, etc., the other polypeptide. The two nucleic acids are said to be operably linked or operably associated. For example, transcription of a nucleic acid is directed by an operably linked promoter; post-transcriptional processing of a nucleic acid is directed by an operably linked processing sequence; translation of a nucleic acid is directed by an operably linked translational regulatory sequence such as a translation initiation sequence; transport, stability, or localization of a nucleic acid or polypeptide is directed by an operably linked transport or localization sequence such as a secretion signal sequence; and post-translational processing of a polypeptide is directed by an operably linked processing sequence. In certain embodiments, a first nucleic acid sequence that is operably linked to a second nucleic acid sequence, or a first polypeptide that is operatively linked to a second polypeptide, is covalently linked, either directly or indirectly, to such a sequence, although any effective three-dimensional association is acceptable. One of ordinary skill in the art will appreciate that multiple nucleic acids, or multiple polypeptides, may be operably linked or associated.

“Plurality” means more than one.

The term “polymorphism” refers to the occurrence of two or more alternative genomic DNA sequences or alleles in a population. Either of the sequences themselves, or the site at which they occur, may also be referred to as a polymorphism. A “single nucleotide polymorphism” or “SNP” is a polymorphism that exists at a single nucleotide position.

A “polymorphic site” or “polymorphic position” is a location at which differences in genomic DNA sequence exist among members of a population. While in general the polymorphic sites described herein are single nucleotides, the term is not limited to sites that are only one nucleotide in length. An “ambiguity code” such as that described in U.S. Ser. No. 10/505,936 may be used to describe a polymorphic site. A “polymorphic region” is a region of genomic DNA that includes one or more polymorphic sites.

A “polymorphic variant” is any of the alternate sequences that may exist at a polymorphic site among members of a population. For purposes of the present invention, the term “population” may refer to the population of the world, or to a subset thereof Typically, for the various methods described herein (e.g., diagnostic methods, methods for identifying causative mutations, etc.), it will be of interest to determine which polymorphic variant(s) is/are present in a individual, among multiple polymorphic variants that exist within a population.

“Polypeptide”, as used herein, refers to a polymer of amino acids. A protein is a molecule composed of one or more polypeptides. A peptide is a relatively short polypeptide, typically between about 2 and 60 amino acids in length. The terms “protein”, “polypeptide”, and “peptide” are used interchangeably herein. Polypeptides as used herein may contain only amino acids that are naturally found in proteins, although non-natural amino acids (e.g., compounds that do not occur in nature but that can be incorporated into a polypeptide chain) and/or amino acid analogs as are known in the art may alternatively be employed. Non-naturally occurring amino acids, amino acids which only occur naturally in an unrelated biological system, modified amino acids whether naturally occurring or non-natural can be used. One or more of the amino acids in a polypeptide may be modified, for example, by the addition of one or more chemical entities such as a carbohydrate group, a phosphate group, a lipid group, etc. Non-limiting examples include a farnesyl group, an isofarnesyl group, a fatty acid group, a glycosyl group, an acetyl group, etc. Polypeptides may contain a linker for conjugation, functionalization, or other modification, etc. In some embodiments, the modifications lead to a more stable polypeptide (e.g., greater half-life in vivo). Exemplary modifications may include cyclization of the peptide, the incorporation of D-amino acids, etc. In certain embodiments the modifications do not substantially interfere with a desired biological activity of the polypeptide.

Polypeptides may, for example, be purified from natural sources, produced in vitro or in vivo in suitable expression systems using recombinant DNA technology in suitable expression systems (e.g., by recombinant host cells or in transgenic animals or plants), synthesized through chemical means such as conventional solid phase peptide synthesis and/or methods involving chemical ligation of synthesized peptides (see, e.g., Kent, S., J Pept Sci., 9(9):574-93, 2003), or any combination of the foregoing. A polypeptide may comprise one or more chemical ligation sites as described, for example, in U.S. Pub. No. 20040115774. In certain embodiments, one or more polypeptides are modified with a polymer using one or more of the methods described or referenced therein. The term “amino acid sequence” or “polypeptide sequence” as used herein refers to the polypeptide material itself and is not restricted to the sequence information (i.e. the succession of letters or three letter codes chosen among the letters and codes used as abbreviations for amino acid names) that biochemically characterizes a polypeptide.

“Probes” or “primers”, as used herein, typically refer to oligonucleotides that hybridize in a sequence-specific manner to a complementary nucleic acid molecule. The term “primer” in particular generally refers to a single-stranded oligonucleotide that can act as a point of initiation of template-directed DNA synthesis using methods including but not limited to PCR (polymerase chain reaction) or LCR (ligase chain reaction) under appropriate conditions (e.g., in the presence of four different nucleoside triphosphates and a polymerization agent, such as DNA polymerase, RNA polymerase or reverse transcriptase, DNA ligase, etc.) in an appropriate buffer solution containing any necessary cofactors and at a suitable temperature. A primer pair may be designed to amplify a region of DNA using PCR. Such a pair will include a “forward” and a “reverse” primer that hybridize to complementary strands of a DNA molecule at locations that delimit a region to be amplified.

Typically, a probe or primer will comprise a region of nucleotide sequence that hybridizes to at least about 8, more often at least about 10 to 15, typically about 20-25, and frequently about 40, 50 or 75, consecutive nucleotides of a target nucleic acid. In certain embodiments, a probe or primer comprises 100 or fewer nucleotides, from 6 to 50 nucleotides, or from 12 to 30 nucleotides. In certain embodiments, the probe or primer is at least 70%, 80%, 90%, 95% or more identical to the contiguous nucleotide sequence or to the complement of the contiguous nucleotide sequence. In certain embodiments, a probe or primer is capable of selectively hybridizing to a target contiguous nucleotide sequence or to the complement of the contiguous nucleotide sequence. In certain embodiments, a probe or primer further comprises a label. For example, a label may be a radioisotope, fluorescent compound, enzyme, or enzyme co-factor.

Oligonucleotides that exhibit differential or selective binding to polymorphic sites may readily be designed by one of ordinary skill in the art. For example, an oligonucleotide that is perfectly complementary to a sequence that encompasses a polymorphic site (e.g., a sequence that includes the polymorphic site within it or at one or the other end) will generally hybridize preferentially to a nucleic acid comprising that sequence as opposed to a nucleic acid comprising an alternate polymorphic variant.

The term “primer” refers to a single-stranded oligonucleotide which acts as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer solution containing any necessary cofactors and at a suitable temperature. The length of a primer depends on the intended use of the primer, but typically ranges from approximately 10 to approximately 30 nucleotides. Short primer molecules generally require lower temperatures to form sufficiently stable hybrid complexes with the template. A primer need not be perfectly complementary to the template but should be sufficiently complementary to hybridize with it. One of ordinary skill in the art will be aware of other constraints that should be considered when designing primers.

The term “regulatory element” or “regulatory sequence” in reference to a nucleic acid is generally used herein to describe a portion of nucleic acid that directs or controls one or more steps in the expression (particularly transcription, but in some cases other events such as splicing or other processing) of nucleic acid sequence(s) with which it is operatively linked. The term includes promoters and can also refer to enhancers, silencers, and other transcriptional control elements. Promoters are regions of nucleic acid that include a site to which RNA polymerase binds before initiating transcription and that are typically necessary for even basal levels of transcription to occur. Generally such elements comprise a TATA box. Enhancers are regions of nucleic acid that encompass binding sites for protein(s) that elevate transcriptional activity of a nearby or distantly located promoter, typically above some basal level of expression that would exist in the absence of the enhancer. In some embodiments, regulatory sequences may direct constitutive expression of a nucleotide sequence, e.g., expression may occur in most or all cell types and/or under most or all conditions. In some embodiments, regulatory sequences may direct cell or tissue-specific and/or inducible expression. For example, expression may be induced by the presence or addition of an inducing agent such as a hormone or other small molecule, by an increase in temperature, etc. Regulatory elements may also inhibit or decrease expression of an operatively linked nucleic acid. Regulatory elements that behave in this manner will be referred to herein as “negative regulatory elements.

In general, the level of expression may be determined using standard techniques for measuring mRNA or protein. Such methods include Northern blotting, in situ hybridization, RT-PCR, sequencing, immunological methods such as immunoblotting, immunodetection, or fluorescence detection following staining with fluorescently labeled antibodies, oligonucleotide or cDNA microarray or membrane array, protein array analysis, mass spectrometry, etc. A convenient way to determine expression level is to place a nucleic acid that encodes a readily detectable marker (e.g., a fluorescent or luminescent protein such as green fluorescent protein or luciferase, an enzyme such as alkaline phosphatase, etc.) in operable association with the regulatory element in an expression vector, introduce the vector into a cell type of interest or into an organism, maintain the cell or organism for a period of time, and then measure expression of the readily detectable marker, taking advantage of whatever property renders it readily detectable (e.g., fluorescence, luminescence, alteration of optical property of a substrate, etc.). Comparing expression in the absence and presence of the regulatory element indicates the degree to which the regulatory element affects expression of an operatively linked sequence.

As used herein, a “sample” obtained from a individual may include, but is not limited to, any or all of the following: a cell or cells, a portion of tissue, blood, serum, ascites, urine, saliva, amniotic fluid, cerebrospinal fluid, and other body fluids, secretions, or excretions. The sample may be a tissue sample obtained, for example, from skin, muscle, buccal or conjunctival mucosa, placenta, gastrointestinal tract or other organs. A sample of DNA from fetal or embryonic cells or tissue can be obtained by appropriate methods, such as by amniocentesis or chorionic villus sampling. The term “sample” also includes any material derived by isolating, purifying, and/or processing a sample as previously defined. Derived samples may include nucleic acids or proteins extracted from the sample or obtained by individualing the sample to techniques such as amplification or reverse transcription of mRNA, etc.

“Specific binding” generally refers to a physical association between a target molecule and a binding molecule such as, for example, physical association between a polypeptide and an antibody or ligand. The association is typically dependent upon the presence of a particular structural feature of the target such as an antigenic determinant or epitope recognized by the binding molecule. For example, if an antibody is specific for epitope A, the presence of a polypeptide containing epitope A or the presence of free unlabeled A in a reaction containing both free labeled A and the binding molecule that binds thereto, will reduce the amount of labeled A that binds to the binding molecule. It is to be understood that specificity need not be absolute but generally refers to the context in which the binding occurs. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies or ligands having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target versus the affinity of the binding molecule for other targets, e.g., competitors. If a binding molecule exhibits a high affinity for a target molecule that it is desired to be detected and low affinity for nontarget molecules, the antibody will likely be an acceptable reagent. Once the specificity of a binding molecule is established in one or more contexts, it may be employed in other contexts without necessarily re-evaluating its specificity. Binding of two or more molecules may be considered specific if the affinity (equilibrium dissociation constant, Kd) is at least 10⁻³ M, 10⁻⁴ M, 10⁻⁵ M, e.g., 10⁻⁶ M, 10⁻⁷ M, 10⁻⁸ M, or 10⁻⁹ M or lower under the conditions tested, e.g., under physiological conditions.

The term “statin” is intended to embrace all inhibitors of the enzyme 3-hydroxy-3-methylglutaryl coenzyme A (HMG-CoA) reductase. Statins specifically inhibit the enzyme HMG-CoA reductase which catalyzes the rate limiting step in cholesterol biosynthesis. Known statins include Atorvastatin, Cerivastatin, Fluvastatin, Lovastatin, Pravastatin and Simvastatin.

“Therapeutic regimen” as used herein refers to treatments aimed at the elimination or amelioration of symptoms and events associated cardiovascular disease. Such treatments include without limitation one or more of alteration in diet, lifestyle, and exercise regimen; invasive and noninvasive surgical techniques such as atherectomy, angioplasty, and coronary bypass surgery; and pharmaceutical interventions, such as administration of ACE inhibitors, angiotensin II receptor antagonists, diuretics, alpha-adrenoreceptor antagonists, cardiac glycosides, phosphodiesterase inhibitors, beta-adrenoreceptor antagonists, calcium channel blockers, HMG-CoA reductase inhibitors, imidazoline receptor blockers, endothelin receptor blockers, organic nitrites, and modulators of protein function of genes listed in Tables 1 and 2. Interventions with pharmaceutical agents not yet known that are useful therapeutically in individuals with particular genotypes associated with cardiovascular disease, e.g., individuals who possess one or more of the CVDA polymorphic variants or haplotypes described herein, are also encompassed. It is contemplated, for example, that patients who are candidates for a particular therapeutic regimen will be screened for genotypes that correlate with responsivity to that particular regimen.

“Treating”, as used herein, can generally include reversing, alleviating, reducing, inhibiting the progression of, or reducing the likelihood of the disease, disorder, or condition to which such term applies, or one or more symptoms or manifestations of such disease, disorder or condition. “Preventing” refers to causing a disease, disorder, condition, or symptom or manifestation of such, or worsening of the severity of such, not to occur.

### DETAILED DESCRIPTION OF CERTAIN EMBODIMENTS

Cardiovascular Disease Associated Polymorphisms and Polymorphic Variants

The present invention is based in part on results of an association study that was performed to identify genetic variations that are associated with an increased risk of cardiovascular disease, particularly acute coronary events such as MI. The association study compared the frequencies of polymorphic variants at a large number of different polymorphic sites between a group of individuals with a history of MI (“cases”) and a group of individuals without a history of MI but having an otherwise similar profile of classical risk factors for cardiovascular disease (“controls”, also referred to as “healthy” individuals).

The study described herein identified SNPs for which the frequencies of the polymorphic variants differed between these two groups of individuals. In general, those polymorphic variants that occur at higher frequency in individuals who are affected by a disease are said to be associated with the disease or condition, and an association is said to exist between the polymorphic variant and the disease or condition. In certain embodiments, the invention provides SNPs for which the frequencies of the polymorphic variants differ between those individuals who have or are susceptible to development or occurrence of a cardiovascular disease or major coronary event such as a myocardial infarction or sudden cardiac death and those individuals who do not have or are not susceptible (or exhibit decreased susceptibility) to development or occurrence of a cardiovascular disease or major coronary event.

Polymorphic variants that were found to be significantly associated with an acute coronary event, including MI, are referred to as “cardiovascular disease associated (CVDA) polymorphic variants”, and a SNP at which such a polymorphic variant exists is referred to as a “CVDA SNP” or “CVDA polymorphism”. Such SNPs are also said to be associated with cardiovascular disease although one of ordinary skill in the art will appreciate that typically only one of the polymorphic variants that can exist at the polymorphic position of the SNP is found at higher frequency among individuals who have or are susceptible to development of cardiovascular disease or major coronary event. In certain embodiments, the invention provides both polymorphic variants that are associated with cardiovascular disease and polymorphic variants that are not associated with cardiovascular disease.

Certain of the CVDA polymorphisms are located within genes. A gene that harbors one or more CVDA SNPs will be referred to herein as a “CVD associated gene” or “CVDA gene”, regardless of the actual function of the gene. The CVDA genes identified in the present invention include a large number of genes that had not been previously linked to CVD. A CVDA gene will typically encode a polypeptide, although it will be understood that CVDA genes are not limited to polypeptide-encoding genes. A polypeptide encoded by a CVDA gene is referred to herein as a CVDA polypeptide. If a CVDA polymorphism occurs within a coding portion of the gene, the polymorphism may result in an alteration in the amino acid sequence of the encoded protein. A SNP at such a location that alters the coding sequence of a protein is referred to as a coding SNP (cSNP). The present invention provides cSNPs wherein the allele that is associated with cardiovascular disease alters the coding sequence of a protein encoded by a CVDA gene. Without wishing to be bound by any theory, genes that include cSNPs are particularly attractive candidates as playing a significant role in cardiovascular disease, e.g., particular alleles of these genes play a causative role in cardiovascular disease or play a protective role. Functionally relevant SNPs can also reside outside protein coding regions, e.g., in the 5′ or 3′ region of a gene (e.g. the promoter or the 3′ untranslated region) and may influence gene expression without affecting the structure of the encoded protein. Without wishing to be bound by any theory, genes that include such functionally relevant SNPs are also attractive candidates as playing a role in CVD.

Each allelic variant of a gene may encode a different polymorphic form of the protein. Since allelic variants of a gene can vary from one another at multiple polymorphic sites, there can be multiple different polymorphic forms of a protein, each reflecting a different combination of polymorphic variants present in the allele that encodes it. Certain aspects of the invention thus provide polymorphic forms of the proteins encoded by the genes identified herein, e.g., genes that include a SNP disclosed herein. The sequence of such proteins will differ from one another at one or more polymorphic positions in the protein, which correspond to the polymorphic positions in the gene.

It will be appreciated that SNPs may be linked to other SNPs located on the same chromosome (linkage disequilibrium) and that such SNPs may be present in haplotypes. For example, it has been shown that SNPs can be linked over significant distances, e.g., 100 kb, or in some cases over more than 150 kb (Reich D. E. et al. Nature 411, 199-204, 2001). In certain aspects, the invention therefore provides haplotypes comprising at least one CVDA polymorphic variant disclosed herein. In certain embodiments, a set of polymorphic variants that constitute a haplotype of the invention is found within a haplotype block. A haplotype block is identified as described in Gabriel, supra. Briefly, 95% confidence bounds on D′ are generated and each comparison is called “strong LD”, “inconclusive” or “strong recombination”. A block is created if 95% of informative (i.e. non-inconclusive) comparisons are “strong LD”.

SNPs that comprise polymorphic variants that are linked to the CVDA polymorphic variants provided herein (referred to herein as “linked SNPs”) can also be used as marker SNPs for cardiovascular disease regardless of whether they are among the SNPs disclosed herein. As in the case of the CVDA SNPs, the frequencies of the polymorphic variants of a linked SNP differ between those individuals who have or are susceptible to development or occurrence of a cardiovascular disease or major coronary event and those and those individuals who do not have or are not susceptible (or exhibit decreased susceptibility) to development or occurrence of a cardiovascular disease or major coronary event. Therefore, one or more polymorphic variants of a linked SNP may be associated with cardiovascular disease and can be used in accordance with certain methods of the present invention.

A large number of SNPs and their chromosomal locations are known in the art and are publicly available in databases such as dbSNP, provided by the National Center for Biotechnology Information (NCBI) (available at the website having the URL www.ncbi.nlm.nih.gov/projects/SNP/), the International HapMap Project (available at the website having the URL www.hapmap.org/), etc. These databases provide a wide variety of information including, e.g., the identity of the nucleotide at each polymorphic position and whether it is a major or minor allele, the sequence surrounding each polymorphic position, chromosome and chromosomal location, gene names and identifiers for SNPs that lie within genes, biological annotation if available, etc. Thus one of ordinary skill in the art, provided with a SNP identifier can readily determine the exact sequence of the polymorphic variants at the polymorphic position and the surrounding sequence. Furthermore, comprehensive information about the particular SNPs that are present on the gene chips that were used to identify the SNPs of the present invention is available at the Affymetrix NetAffx™ Analysis Center (available at the web site having the URL www.affymetrix.com/analysis/index.affx).

Tables 1 and 2 provides identifying information for CVDA SNPs of the present invention. Each row of Tables 1 and 2 identifies a SNP that is encompassed within the present invention. Each row describing a SNP contains 19 columns containing data. In addition to information identifying the SNP, certain additional information related to the SNP, including the allele frequencies determined as described herein, is provided in Tables 1 and 2s. General information about the SNP, such as the chromosome, chromosomal location, gene name, major and minor alleles, etc., as well as additional information, can readily be found in dbSNP, HapMap, or the Affymetrix NetAffx™ Analysis Center by searching using the SNP ID. Specifically, a search box is provided at the secure website having the URL www.affymetrix.com/analysis/netaffx/quickquery.affx?mapping=true, into which the user can enter a SNP ID and perform a search to retrieve information related to the SNP. For purposes of convenience some of this information is included in Table 1.

The contents of Tables 1 and 2 are now described by column, beginning on the left and moving to the right. The data set was divided based on chromosome due to the large amount of data. Thus Tables 1 and 2 group SNPs by chromosome. The first column of Tables 1 and 2 (column heading “Name”) is the Affymetrix ID for the SNP. The second column of Tables 1 and 2 (column heading “Major_Allele_{—}1”) gives the base (nucleotide) that is present in the major allele in the cases (i.e., the more frequent allele in the cases). The third column of Tables 1 and 2 (column heading “Major_Allele_{—}2”) gives the base (nucleotide) that is present in the major allele in the controls (i.e., the more frequent allele in the controls).

For example, the first row of data in Tables 1 and 2 (identified by Affymetrix ID SNP_A-2081399) contains “C” under both the headings “Major_Allele_{—}1” and “Major_Allele_{—}2”. This indicates that the major allele in both the cases and the controls contained a C at the polymorphic position. As another example, the row identified by Affymetrix ID SNP_A-1856955 contains “G” under the heading “Major_Allele_{—}1” and “A” under the heading “Major_Allele_{—}2”. This indicates that the major allele in the cases contained a G at the polymorphic position, and the major allele in the controls contained an A at the polymorphic position.

The fourth and fifth columns of Tables 1 and 2 (column headings “Case_Major” and “Case_Minor”) present the actual counts for alleles A and B in cases. The sixth and seventh columns of Tables 1 and 2 (column headings “Control_Major” and “Control_Minor”) present the actual counts for alleles A and B in controls. For example, in the first row of data in Table 1 (SNP A-2081399), allele A was detected 192 times in cases, while allele B was detected 182 times in cases. Similarly, allele A was detected 234 times in controls, while allele B was detected 166 times in controls. It is noted that the numbers do not always add up to the same value because it was not possible to accurately identify the allele in some instances. The allele frequencies are readily computed from the data in this column. Consider the following example from the first row of data in Table 1 (SNP_A-2081399):

| Case ratio | Control ratio |
|---|---|
| 192:182 | 234:166 |

In the cases, allele A has a frequency of 192/374 (˜51.3%) and allele B has a frequency of 182/374 (˜48.7%), while in the controls allele A has a frequency of 234/400 (˜58.5%) and allele B has a frequency of 166/400 (˜41.5%). Thus it is evident that the allele frequencies differ between the cases and controls. The difference between the allele frequencies is statistically significant (as shown by the Chi square value of 4.008 and the P value of 0.0453), with allele B occurring more frequently in cases than in controls (i.e., allele B has a frequency of ˜48.7% in cases and ˜41.5% in controls) and allele A occurring more frequently in controls than in cases (i.e., allele A has a frequency of ˜58.5% in controls and ˜51.3% in cases). Therefore, allele B is the variant that is associated with cardiovascular disease and is a CVDA polymorphic variant of this particular CVDA polymorphism, in accordance with the terminology described above. It should be noted that both alleles of each SNP are informative with respect to CVD; thus detection of either allele, or both, of each of the SNPs identified herein, is one non-limiting aspect of this invention.

The eighth column of Tables 1 and 2 (column heading “Chi Square”) presents the Pearson's Chi-square for allele frequencies.

The ninth column of Tables 1 and 2 (column heading “p_value”) presents the probability value (significance) for each Chi square/observation.

The tenth column of Tables 1 and 2 (column heading “dbSNP_RS_ID”) is the SNP annotation (i.e., the SNP name) as annotated in public databases (dbSNP at NCBI or HapMap), where available. Each SNP identifier begins with the letters “rs“and is followed by a string of numbers.

The eleventh column of Tables 1 and 2 (column heading “Chromosome”) presents the chromosome on which the SNP is located.

The twelfth column of Tables 1 and 2 (column heading “Physical_Position”) presents the physical position of the SNP in the human genome.

The thirteenth column of Tables 1 and 2 (column heading “Cytoband”) lists the chromosomal arm and band at which the SNP is located.

The fourteenth column of Tables 1 and 2 (column heading “Flank”) presents a portion of the genomic sequence that surrounds and includes the SNP. The polymorphic site, and the alternative nucleotides that are present in different polymorphic variants, are indicated near the center of the sequence in brackets and upper case letters.

The fifteenth and sixteenth columns of Tables 1 and 2 (column headings “Allele_A” and “Allele_B” present the alternative nucleotides that are present in different polymorphic variants, with “Allele_A” representing the major allele as assigned by Affymetrix.

The seventeenth column of Tables 1 and 2 (column heading “Associated_Gene”) provides information about the gene or location where the SNP resides, including relevant accession numbers for databases such as GenBank in some cases.

The eighteenth column of Tables 1 and 2 (column heading “Gene_Symbol”) is GENE SYMBOL (the SYMBOL that was assigned to the gene by the Human Genome Project and how it can be located in all relevant GENOME Databases).

The nineteenth column of Tables 1 and 2 (column heading “Label”) provides further explanation of where the SNP resides and repeats part of the information in other columns such as GENE SYMBOL, the rs-number (“dbSNP_RS_ID”); the gene name (or other identifying information, if applicable) followed by cytogenetic chromosomal location (arm and band).

In addition to the SNPs provided in Tables 1 and 2, the present invention provides additional SNPs, e.g., SNPs located at a distance of less than about 100-150 kB of any of the CVDA SNPs of the present invention. These SNPs may readily be obtained from dbSNP, HapMap, or the Affymetrix NetAffx™ Analysis Center resources by one of ordinary skill in the art and are therefore not explicitly identified by SNP identifier herein. The SNP may be tested in a variety of ways to determine whether a polymorphic variant of the SNP is associated with cardiovascular disease. For example, the SNP may be tested as described in Example 1. In some instances, a polymorphic variant of the additional SNP may already be known to be in LD with a CVDA polymorphic variant described herein. Therefore, in certain embodiments, the invention provides a linked SNP for which the frequencies of the polymorphic variants found at the location of the linked SNP differ between individuals who have or are susceptible to development or occurrence of a cardiovascular disease or event. In certain embodiments, a polymorphic variant of the linked SNP is in strong linkage disequilibrium with a CVDA polymorphic variant. In certain embodiments, such linked SNP is located within 1 kB of the CVDA SNP with which it is linked. In certain embodiments, a linked SNP is located up to 10 kB, up to 20 kB, up to 30 kB, up to 40 kB, up to 50 kB, up to 60 kB, up to 70 kB, up to 80 kB, up to 90 kB, or up to 100 kB away from the CVDA SNP with which it is linked. In certain embodiments, a linked SNP is located within the same gene as the CVDA SNP with which it is linked.

In general, a linked SNP comprises a polymorphic variant that is part of a haplotype that includes at least one CVDA polymorphic variant. In certain embodiments, the invention therefore provides a haplotype comprising at least one CVDA polymorphic variant and a polymorphic variant of at least one linked SNP, wherein the polymorphic variant of the linked SNP is associated with cardiovascular disease. The SNP may be any linked SNP known in the art.

An alternative approach that can be used to identify polymorphic variants that are in LD with CVDA polymorphic variants is to sequence the genomic DNA of a plurality of cases and controls in a region located in the vicinity of a CVDA SNP. For example, a region of any length located within up to approximately 150 kB of the SNP, e.g., a region approximately 1, 5, 10, 20, 50, 100, 150 kB, etc., in length can be sequenced from a plurality of individuals. In certain embodiments, such a region encompasses the CVDA SNP. Nucleotide differences among individuals, e.g., SNPs, are identified. In certain embodiments, such individuals include both cases and controls, although this is not necessary for purposes of simply identifying the SNP. A plurality of cases and controls may be genotyped with respect to the SNPs, and the allele frequencies may be determined. Alleles that are associated with cardiovascular disease, e.g., variants that occur more frequently in a statistically significant sense in individuals with cardiovascular disease may be identified. Each of such polymorphic variants is an additional CVDA polymorphic variant, and a SNP having such a variant is an additional CVDA SNP.

In certain embodiments, the invention therefore provides methods of identifying a polymorphism comprising: sequencing a region of DNA from a plurality of individuals, wherein the region of DNA lies within 150 kB of a CVDA SNP and identifying nucleotide differences in the sequence of the DNA region among individuals. Such differences occur at polymorphic positions, and the various nucleotide sequences present at those positions are polymorphic variants. The polymorphism may be a SNP. Certain methods comprise an additional step of determining whether a polymorphic variant is in LD with a CVDA polymorphic variant. The method can comprise an additional step of determining whether a polymorphic variant is associated with cardiovascular disease.

As mentioned above, and without wishing to be bound by any theory, certain of the CVDA polymorphisms may play a causative role in cardiovascular disease. For example, a particular polymorphic variant associated with cardiovascular disease may have an altered expression level, altered expression pattern, and/or altered functional activity relative to alleles that are not associated with cardiovascular disease, and such alteration may contribute to the development of cardiovascular disease. While not wishing to be bound by any theory, this is particularly likely to be the case for those polymorphisms that lie within genes, and most particularly for those that lie within coding sequences and in which the polymorphic variants result in proteins with differences in amino acid sequence. However, alterations that lie outside genes can, for example, also affect expression and can play a causative role. In certain embodiments, CVDA polymorphic variants do not play a causative role, but are in LD with polymorphic variants that do play such a role. Such non-causative CVDA polymorphic variants may not be direct targets for treatment, but they may nevertheless be of use for diagnostic purposes such as those described herein.

In certain embodiments, polymorphic variants are identified that are associated with CVD in only a subset of individuals, wherein the subset is classified based on a risk assessment based on classical risk factors and/or other markers of cardiovascular status. For example, certain variants may be associated with increased risk in individuals having a risk of <10% but not in individuals having a risk of >20%. Similarly, certain variants may be associated with increased risk in individuals having a risk of >20% but not in individuals having a risk of <10%.

Diagnostic and Prognostic Methods and Reagents

The invention encompasses a variety of methods for determining whether an individual has or is susceptible to development or occurrence of a cardiovascular disease or event, wherein the individual is in need of such determination. By “event” is meant a major coronary event such as a myocardial infarction or sudden cardiac death. In certain embodiments, methods of the present invention comprise steps of: (a) detecting a polymorphic variant of a CVDA polymorphism in the individual; (b) or detecting a polymorphic variant in strong LD with a CVDA polymorphism; or (c) detecting a haplotype comprising a polymorphic variant of the CVDA polymorphism in the individual; or (d) detecting an allele of a gene comprising the polymorphic variant of the CVDA polymorphism in the individual.

Methods described herein are typically practiced on a sample obtained from the individual and the phrase “in the individual” is to be understood in that light. The sample typically contains genetic material, e.g., DNA. Such DNA may be obtained from any cell source, including any cell source. Non-limiting examples of cell sources available in clinical practice include blood cells, buccal cells, cervicovaginal cells, epithelial cells from urine, fetal cells, or any cells present in tissue obtained by biopsy. Cells may also be obtained from body fluids, including without limitation blood, saliva, sweat, urine, cerebrospinal fluid, feces, and tissue exudates at the site of infection or inflammation. DNA is extracted from the cell source or body fluid using any of the numerous methods that are standard in the art. It will be understood that the particular method used to extract DNA will depend on the nature of the source. In some embodiments, inventive methods are practiced on cellular material other than DNA. For example, polymorphisms that lie in genes may be detected in RNA. Polymorphisms that affect expression level may be detected by measuring mRNA or protein level. Furthermore, polymorphisms that alter the coding sequence of a protein may be detected by examining the protein as described elsewhere herein.

“Detecting a polymorphic variant” is used in a broad sense and refers, e.g., to determining the identity of the polymorphic variant by any suitable means, wherein determining the identity means providing sufficient information to determine whether the variant is a variant that has been identified herein as being associated with cardiovascular disease. It will be appreciated that detecting a polymorphic variant can encompass detecting the absence of a different polymorphic variant, and thus determining that a particular variant is absent is one means of determining that a different variant is present. Thus, in certain embodiments, methods of the present invention comprise detecting, in a sample of cells from the subject, the presence or absence of a specific allelic variant, e.g., an allelic variant of one or more polymorphic regions of a gene or an allelic variant in an intergenic region. The allelic differences can be: (i) a difference in the identity of at least one nucleotide or (ii) a difference in the number of nucleotides, which difference can be a single nucleotide or several nucleotides. It will typically be of interest to detect and identify both of the individual's alleles, but this need not be the case.

In certain embodiments, suitable detection methods comprise allele specific hybridization using probes overlapping the polymorphic site and typically having about 5, 10, 20, 25, or 30 nucleotides around the polymorphic region. Examples of probes for detecting specific allelic variants of a CVDA polymorphism are probes comprising at least 10 nucleotides of a nucleotide sequence set forth in Tables 1 and 2, wherein the at least 10 nucleotides include the polymorphic position. In certain embodiments, several probes capable of hybridizing specifically to allelic variants are attached to a solid support, e.g., a “chip”. Oligonucleotides can be bound to a solid support by a variety of processes such as lithography. Probes can be synthesized directly on a chip. For example a chip can hold up to 250,000 oligonucleotides or more (GeneChip, Affymetrix). Hybridization, followed by scanning to determine the position(s) on the array to which a nucleic acid hybridizes, is performed according to standard methods.

Detection of polymorphic variants using chips comprising oligonucleotides, also termed “DNA probe arrays”, “oligonucleotide arrays”, etc., has been known in the art for some time and is described e.g., in Cronin et al., Human Mutation 7:244, 1996 and in Kozal et al., Nature Medicine 2:753, 1996. See also Matsuzaki, H., et al., Genome Research, 14:414-425, 2004, describing use of a high density oligonucleotide array for parallel genotyping of over 10,000 SNPs. The present invention utilized such arrays for the identification of the CVDA SNPs as described in more detail in Example 1.

In certain embodiments, a chip comprises all the allelic variants of at least one genomic region that comprises a polymorphic position. The solid support is then contacted with a test nucleic acid or preparation of nucleic acids (e.g., DNA or RNA extracted from a cell, a specific nucleic acid that has been amplified, etc.) and hybridization to the specific probes is detected. Accordingly, the identity of numerous allelic variants of one or more genes can be identified in a simple hybridization experiment. For example, the identity of the allelic variant of the nucleotide polymorphism of one or more identified SNPs identified in Tables 1 and 2 and that of the allelic variants of a plurality of other polymorphic regions can be determined in a single hybridization experiment.

Arrays can include multiple detection blocks (e.g., multiple groups of probes designed for detection of particular polymorphisms). Such arrays can be used to analyze multiple different polymorphisms. Detection blocks may be grouped within a single array or in multiple, separate arrays so that varying conditions (e.g., conditions optimized for particular polymorphisms) may be used during the hybridization. For example, it may be desirable to provide for the detection of those polymorphisms that fall within G-C rich stretches of a genomic sequence, separately from those falling in A-T rich segments.

Additional description of use of oligonucleotide arrays for detection of polymorphisms can be found, for example, in U.S. Pat. Nos. 5,858,659 and 5,837,832. In addition, to oligonucleotide arrays, cDNA arrays may be used similarly in certain embodiments.

In some detection methods, at least a portion of the nucleic acid is amplified prior to identifying the allelic variant. Amplification can be performed, e.g., by using the polymerase chain reaction (PCR) and/or ligase chain reaction (LCR), according to methods known in the art. In certain embodiments, genomic DNA of a cell is exposed to two PCR primers and amplification for a number of cycles sufficient to produce the required amount of amplified DNA. In certain embodiments, the primers are located between 40 and 350 base pairs apart. Primers for amplifying regions of DNA comprising the CVDA polymorphic sites may readily be designed based on the human genome sequence. Such primers will typically be complementary to portions of DNA that flank the polymorphic site. See e.g., PCR Primer: A Laboratory Manual, Dieffenbach, C. W. and Dveksler, G. S. (Eds.); PCR Basics: From Background to Bench, Springer Verlag, 2000; M. J. McPherson, et al; Mattila et al., Nucleic Acids Res., 19:4967 (1991); Eckert et al., PCR Methods and Applications, 1:17 (1991); PCR (eds. McPherson et al., IRL Press, Oxford); and U.S. Pat. No. 4,683,202. Guidelines for selecting primers for PCR amplification are well known in the art. See, e.g., McPherson, M., et al., PCR Basics: From Background to Bench, Springer-Verlag, 2000. A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis. 53711).

Alternative amplification methods include: self sustained sequence replication (Guatelli, J. C. et al., 1990, Proc. Natl. Acad. Sci. U.S.A. 87:1874-1878), transcriptional amplification system (Kwoh, D. Y. et al., 1989, Proc. Natl. Acad. Sci. U.S.A. 86:1173-1177), Q-Beta Replicase (Lizardi, P. M. et al., 1988, Bio/Technology 6:1197), or any other nucleic acid amplification method. For example, other amplification methods that may be employed include the ligase chain reaction (LCR) (Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Nat. Acad. Sci. USA, 87:1874 (1990)), and nucleic acid based sequence amplification (NASBA).

Amplification is typically followed by or occurs simultaneously with the detection of the amplified molecules using techniques well known to those of skill in the art. Detection schemes involving amplification can be especially useful for the detection of nucleic acid molecules if such molecules are present in relatively or absolutely low numbers.

The invention encompasses the use of real-time PCR, e.g., the 5′ nuclease allelic discrimination or TaqMan® assay (Livak, K. J., et al., Nature Genet. 9: 341-342, 1995; Ranade, K., et al., Genome Research, Vol. 11, Issue 7, 1262-1268, 2001) for high-throughput genotyping. According to this method, the region flanking the polymorphism, typically 100 base pairs, is amplified in the presence of two probes each specific for one or the other allele. Probes have a fluor, also called a “reporter,” at the 5′ end but do not fluoresce when free in solution because they have a “quencher” at the 3′ end that absorbs fluorescence from the reporter. During PCR, the Taq polymerase encounters a probe specifically base-paired with its target and unwinds it. The polymerase cleaves the partially unwound probe and liberates the reporter fluor from the quencher, thereby increasing net fluorescence. The presence of two probes, each labeled with a different fluor, allows one to detect both alleles in a single tube. Moreover, because probes are included in the PCR, genotypes are determined without any post-PCR processing, a feature that is unavailable with most other genotyping methods (see Landegren, U., et al., Genome Res. 8: 769-776, 1998, for a review). Other methods for performing real-time PCR, e.g., using molecular beacons or scorpions could also be used. In certain embodiments, an Invader® cleavage assay is used for detecting one or more polymorphic variants. See, e.g., Lyamichev, V., et al., Nature Biotechnol. 17: 292-296, 1999. Minisequencing on oligonucleotide arrays offers another approach (Pastinen, T., et al., Genome Res. 7: 606-614, 1997).

The invention thus provides a variety of probes and primers of use for detecting a polymorphic variant of a polymorphism listed in Tables 1 and 2. In certain embodiments the probe or primer comprises a nucleotide sequence that hybridizes to at least about 8, more often at least about 10 to 15, typically about 20-25, and frequently about 40, 50 or 75, consecutive nucleotides of a target nucleic acid molecule, e.g., a nucleic acid molecule that comprises a CVDA polymorphism. In certain embodiments, a probe or primer comprises 100 or fewer nucleotides, from 6 to 50 nucleotides, or from 12 to 30 nucleotides. In certain embodiments, a probe or primer is at least 70%, 80%, 90%, 95% or more identical to the contiguous nucleotide sequence or to the complement of the contiguous nucleotide sequence. In certain embodiments, a preferred probe or primer is capable of selectively hybridizing to a target contiguous nucleotide sequence or to the complement of the contiguous nucleotide sequence. According to certain embodiments, a probe or primer further comprises a label, for example by incorporating a radioisotope, fluorescent compound, enzyme, enzyme co-factor, mass tag (for detection using mass spectrometry), etc.

According to certain embodiments, allele specific primers and/or probes correspond exactly with the allele to be detected (e.g., they are identical in sequence or perfectly complementary to a portion of DNA that encompasses the polymorphic site, wherein the site contains any of the possible variants), but derivatives thereof are also provided wherein, for example, about 6-8 of the nucleotides at the 3′, terminus correspond to (e.g., are identical in sequence or perfectly complementary to) the allele to be detected and wherein up to 10, such as up to 8, 6, 4, 2 or 1 of the remaining nucleotides may be varied without significantly affecting the properties of the primer or probe.

The invention further provides a set of oligonucleotide primers, wherein the primers terminate adjacent to a polymorphic site of a CVDA polymorphism, or wherein the primers terminate adjacent to a polymorphic site of a CVDA polymorphism. Such primers are useful, for example, in performing fluorescence polarization template-directed dye-terminator incorporation, as described below. In certain embodiments, the invention provides oligonucleotide primers that terminate immediately adjacent to the polymorphic sites present in the CVDA SNPs identified in Tables 1 and 2.

In certain embodiments, the invention provides, for each of these polymorphisms, a primer that terminates at the nucleotide position immediately adjacent to a polymorphic site on the 3′ side and extends at least 8 and less than 100 nucleotides in the 5′ direction from this site. It is noted that the foregoing includes two classes of primers, having sequences representing both DNA strands. According to certain embodiments, the primer extends at least 10, at least 12, at least 15, or at least 20 nucleotides in the 5′ direction. According to certain embodiments, the primer extends less than 80, less than 60, less than 50, less than 40, less than 30, or less than 20 nucleotides in the 5′ direction. The invention further provides primers that terminate and extend similarly for any polymorphic site of a CVDA SNP, or a polymorphic site linked to such a SNP.

In general, primers and probes of the present invention may be made using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993. According to certain embodiments, the primer(s) and/or probes are labeled to facilitate detection.

Primers and probes of the present invention may be conveniently provided in sets, e.g., sets capable of determining which polymorphic variant(s) is/are present among some or all of the possible polymorphic variants that may exist at a particular polymorphic site. The sets may include allele-specific primers or probes and/or primers that terminate immediately adjacent to a polymorphic site. Multiple sets of primers and/or probes, capable of detecting polymorphic variants at a plurality of polymorphic sites may be provided.

Oligonucleotides that exhibit differential or selective binding to polymorphic sites may readily be designed by one of ordinary skill in the art. For example, an oligonucleotide that is perfectly complementary to a sequence that encompasses a polymorphic site (e.g., a sequence that includes the polymorphic site within it or at one or the other end) will generally hybridize preferentially to a nucleic acid comprising that sequence as opposed to a nucleic acid comprising an alternate polymorphic variant

In some embodiments, any of a variety of sequencing reactions known in the art can be used to directly sequence at least a portion of genomic DNA and detect allelic variants. The sequence can be compared with the sequences of known allelic variants to determine which one(s) are present in the sample. Exemplary sequencing reactions include those based on techniques developed by Maxam and Gilbert, Proc. Natl. Acad Sci USA, 74:560, 1977 or Sanger, Proc. Nat. Acad. Sci 74:5463, 1977. It is also contemplated that any of a variety of automated sequencing procedures may be utilized when performing the subject assays (Biotechniques 19:448, 1995; Venter, et al., Science, 291:1304-1351, 2001; Lander, et al., Nature, 409:860-921, 2001), including sequencing by mass spectrometry (see, for example, U.S. Pat. No. 5,547,835 and international patent application Publication Number WO 94/16101, entitled DNA Sequencing by Mass Spectrometry by H. Koster; U.S. Pat. No. 5,547,835 and international patent application Publication Number WO 94/21822 entitled “DNA Sequencing by Mass Spectrometry Via Exonuclease Degradation” by H. Koster), and U.S. Pat. No. 5,605,798 and International Patent Application No. PCT/US96/03651 entitled DNA Diagnostics Based on Mass Spectrometry by H. Koster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”, and in Melamede, U.S. Pat. No. 4,863,849; Cheeseman, U.S. Pat. No. 5,302,509, Tsien et al, International application WO 91/06678; Rosenthal et al, International application WO 93/21340; Canard et al, Gene, 148: 1-6 (1994); Metzker et al, Nucleic Acids Research, 22: 4259-4267 (1994) and U.S. Pat. Nos. 5,740,341 and 6,306,597.

In some cases, the presence of a specific allele can be shown by restriction enzyme analysis. For example, a specific nucleotide polymorphism can result in a nucleotide sequence comprising a restriction site which is absent from the nucleotide sequence of another allelic variant. Additionally or alternately, a specific nucleotide polymorphism can result in the elimination of a nucleotide sequence comprising a restriction site which is present in the nucleotide sequence of another allelic variant.

In certain embodiments, alterations in electrophoretic mobility are used to identify the allelic variant. For example, single strand conformation polymorphism (SSCP) may be used to detect differences in electrophoretic mobility between two similar nucleic acids (Orita et al., Proc Natl. Acad. Sci USA 86:2766, 1989, see also Cotton, Mutat Res 285:125-144, 1993; and Hayashi, Genet Anal Tech Appl 9:73-79, 1992). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In certain embodiments, the subject method utilizes heteroduplex analysis to separate double stranded heteroduplex molecules on the basis of changes in electrophoretic mobility (Keen et al., Trends Genet 7:5, 1991).

In certain embodiments, the identity of an allelic variant of a polymorphic region is assayed using denaturing gradient gel electrophoresis (“DGGE”). DGGE comprises analyzing the movement of a nucleic acid comprising the polymorphic region in polyacrylamide gels containing a gradient of denaturant (DGGE) (Myers et al., Nature 313:495, 1985). When DGGE is used as the method of analysis, DNA may be modified to insure that it does not completely denature, for example by adding a GC clamp of approximately 40 bp of high-melting GC-rich DNA by PCR. In certain embodiments, a temperature gradient is used in place of a denaturing agent gradient to identify differences in the mobility of control and sample DNA (Rosenbaum and Reissner, Biophys Chem 265:1275, 1987).

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al., Nature 324:163, 1986); Saiki et al., Proc. Natl Acad. Sci USA 86:6230, 1989; and Wallace et al., Nucl. Acids Res. 6:3543, 1979). Such allele specific oligonucleotide hybridization techniques may be used for the simultaneous detection of several nucleotide changes in different polymorphic regions of DNA. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

In certain embodiments, allele specific amplification technology which depends on selective PCR amplification may be used. Oligonucleotides used as primers for specific amplification may carry the allelic variant of interest in the center of the molecule (so that amplification depends on differential hybridization) (Gibbs et al., Nucleic Acids Res. 17:2437-2448, 1989) or at the extreme 3′ end of one primer where, under appropriate conditions, mismatch can prevent, or reduce polymerase extension (Prossner, Tibtech 11:238, 1993; Newton et al., Nucl. Acids Res. 17:2503, 1989). This technique is also termed “PROBE” for Probe Oligo Base Extension. In addition, it may be desirable to introduce a novel restriction site in the region of the mutation to create cleavage-based detection (Gasparini et al., Mol. Cell Probes 6:1, 1992).

In certain embodiments, identification of the allelic variant is carried out using an oligonucleotide ligation assay (OLA), as described, e.g., in U.S. Pat. No. 4,998,617 and in Landegren, U. et al., Science 241:1077-1080, 1988. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927, 1990. In this method, PCR is used to achieve the exponential amplification of target DNA, which is then detected using OLA.

Several techniques based on this OLA method have been developed and can be used to detect specific allelic variants of a polymorphic region. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (Nucleic Acids Res 24: 3728, 1996), OLA combined with PCR permits typing of two alleles in a single microtiter well. By marling each of the allele-specific primers with a unique hapten, e.g. digoxigenin or fluorescein, each LA reaction can be detected by using hapten specific antibodies that are labeled with different enzyme reporters, alkaline phosphatase or horseradish peroxidase. This system permits the detection of the two alleles using a high throughput format that leads to the production of two different colors.

Additionally or alternatively, the invention provides further methods of use for detecting single nucleotide polymorphisms. Because single nucleotide polymorphisms constitute sites of variation flanked by regions of invariant sequence, their analysis requires no more than the determination of the identity of the single nucleotide present at the site of variation. Several methods have been developed to facilitate the analysis of such single nucleotide polymorphisms.

In certain embodiments, a single nucleotide polymorphism can be detected by using a specialized exonuclease-resistant nucleotide, as disclosed, e.g., in Mundy, C. R. (U.S. Pat. No. 4,656,127). According to such embodiments, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction.

In certain embodiments, a solution-based method is used for determining the identity of the nucleotide of a polymorphic site. Cohen, D. et al. (French Patent 2,650,840; PCT App. No. WO91/02087). As in the Mundy method of U.S. Pat. No. 4,656,127, a primer may be employed that is complementary to allelic sequences immediately 3′ to a polymorphic site. Such a method determines the identity of the nucleotide of that site using labeled dideoxynucleotide derivatives, which, if complementary to the nucleotide of the polymorphic site will become incorporated onto the terminus of the primer.

An alternative method is described by Goelet, P. et al. (PCT App. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT App. No. WO91/02087) the method of Goelet, P. et al. is preferably a heterogeneous phase assay, in which the primer or the target molecule is immobilized to a solid phase.

A variety of primer-guided nucleotide incorporation procedures for assaying polymorphic sites in DNA have been described (Komher, J. S. et al., Nucl. Acids. Res. 17:7779-0.7784, 1989; Sokolov, B. P., Nucl. Acids Res. 18:3671, 1990; Syvanen, A.-C., et al., Genomics 8:684-692, 1990, Kuppuswamy, M. N. et al., Proc. Natl. Acad. Sci. (U.S.A.) 88:1143-1147, 1991; Prezant, T. R. et al., Hum. Mutat. 1:159-164, 1992; Ugozzoli, L. et al., GATA 9:107-112, 1992; Nyren, P. et al., Anal. Biochem. 208:171-175, 1993). These methods rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

In certain embodiments, fluorescence polarization template-directed dye-terminator incorporation (FP-TDI) is used to determine which of multiple polymorphic variants of a polymorphism is present in a subject. This method is based on template-directed primer extension and detection by fluorescence polarization. According to this method, amplified genomic DNA containing a polymorphic site is incubated with oligonucleotide primers (designed to hybridize to the DNA template adjacent to the polymorphic site) in the presence of allele-specific dye-labeled dideoxyribonucleoside triphosphates and a commercially available modified Taq DNA polymerase. The primer is extended by the dye-terminator specific for the allele present on the template, increasing ˜10-fold the molecular weight of the fluorophore. At the end of the reaction, the fluorescence polarization of the two dye-terminators in the reaction mixture is analyzed directly without separation or purification. Such a homogeneous DNA diagnostic method has been shown to be highly sensitive and specific and is suitable for automated genotyping of large number of samples. (Chen, X., et al., Genome Research, Vol. 9, Issue 5, 492-498, 1999). Note that rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

Real-time pyrophosphate DNA sequencing is yet another approach to detection of polymorphisms and polymorphic variants (Alderborn, A., et al., Genome Research, Vol. 10, Issue 8, 1249-1258, 2000). Additional methods include, for example, PCR amplification in combination with denaturing high performance liquid chromatography (dHPLC) (Underhill, P. A., et al., Genome Research, Vol. 7, No. 10, pp. 996-1005, 1997).

In general, it will be often of interest to determine the genotype of a subject with respect to both alleles of the polymorphic site present in the genome. For example, the complete genotype may be characterized as −/−, as −/+, or as +/+, where a minus sign indicates the presence of a particular sequence at the polymorphic site (e.g., the major allele, by which is meant the allele that occurs most frequently in a population), and the plus sign indicates the presence of a different polymorphic variant other than the reference sequence. Other methods simply use the identity of the base present at a polymorphic position. If multiple polymorphic variants exist at a site, this can be appropriately indicated by specifying which ones are present. Any of the detection means above may be used to determine the genotype of a subject with respect to one or both copies of the polymorphism present in the subject's genome.

According to certain embodiments, it is advantageous to employ methods that can detect the presence of multiple polymorphic variants (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to certain embodiments of the invention.

For determining the identity of the allelic variant of a polymorphic region located in the coding region of a gene, methods in addition to those described above can be used. For example, identification of an allelic variant which encodes a variant protein can be performed by using an antibody specifically recognizing the variant protein in, e.g., immunohistochemistry or immunoprecipitation. Antibodies to specific variants proteins can be prepared according to methods known in the art and as described herein. Additionally or alternatively, one can also measure a biological or biochemical activity of a protein, such as binding to a particular molecular target or cell. Suitable binding assays are known in the art.

If a polymorphic region is located in an exon, either in a coding or non-coding region of the gene, the identity of the allelic variant can be determined by determining the molecular structure of the mRNA, pre-mRNA, or cDNA. The molecular structure can be determined using any of the above described methods for determining the molecular structure of the genomic DNA, e.g., sequencing and SSCP.

Methods described herein may be performed, for example, by utilizing prepackaged diagnostic kits, such as those described herein comprising at least a reagent such as a probe or primer nucleic acid described herein, which may be conveniently used, e.g., to determine whether a subject has or is susceptible to development or occurrence of a cardiovascular disease or coronary event associated with a specific gene allelic variant.

As mentioned above, nucleic acids or proteins for use in the above-described diagnostic and prognostic methods can be obtained from any cell type or tissue of a subject. For example, a subject's bodily fluid (e.g. blood) can be obtained by known techniques (e.g. venipuncture) or from human tissues like heart (biopsies, transplanted organs). Alternatively, nucleic acid tests can be performed on dry samples (e.g. hair or skin). Fetal nucleic acid samples for prenatal diagnostics can be obtained from maternal blood as described in International Patent Application No. WO91/07660 to Bianchi. Additionally or alternatively, amniocytes or chorionic villi may be obtained for performing prenatal testing.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., PCR in situ hybridization: protocols and applications, Raven Press, New York, 1992).

In certain embodiments, as mentioned above, the presence of absence of a plurality of polymorphic variants at different polymorphic sites is detected. Thus a genetic profile of an individual may be generated, wherein the genetic profile indicates which allelic variant is present at a plurality of different polymorphisms that are associated with cardiovascular disease.

In some embodiments, the genotype of a large number of individuals exhibiting particular risk factors, markers of cardiovascular status, or response to therapy for cardiovascular disease is determined with respect to one or more polymorphisms by any of the methods described above, and compared with the distribution of genotypes of individuals that have been matched for any of a plurality of characteristics such as age, ethnic origin, and/or any other statistically or medically relevant parameters, who exhibit quantitatively or qualitatively risk factors, markers of cardiovascular status, or response to therapy. “Cardiovascular status” as used herein refers to the physiological status of an individual's cardiovascular system as reflected in one or more markers or indicators. Status markers include without limitation clinical measurements such as, e.g., blood pressure, electrocardiographic profile, and differentiated blood flow analysis as well as measurements of LDL- and HDL-cholesterol levels, other lipids (e.g., TGs) and other well established clinical parameters that are standard in the art. It will be appreciated that status markers and risk factors overlap. Status markers according to the invention also include diagnoses of one or more cardiovascular symptoms or syndromes, such as, e.g., hypertension, acute myocardial infarction, silent myocardial infarction, stroke, and atherosclerosis. It will be understood that a diagnosis of a cardiovascular syndrome made by a medical practitioner encompasses clinical measurements and medical judgment. Status markers further include results of imaging analyses, e.g., magnetic resonance imaging, ultrasound imaging such as Doppler imaging, angiograms, and other means to evaluate the structure of the blood vessel wall, blood flow, and the like. Status markers according to the invention may be assessed using conventional methods well known in the art.

Also included in the evaluation of cardiovascular status are quantitative or qualitative changes in status markers with time, such as would be used, e.g., in the determination of an individual's response to a particular therapeutic regimen. Correlations are achieved using any method known in the art, including nominal logistic regression, chi square tests or standard least squares regression analysis. In this manner, it is possible to establish statistically significant correlations between particular genotypes and particular risk factors, markers of cardiovascular status, or response to therapy for cardiovascular disease (e.g., given in terms of p values). It is further possible to establish statistically significant correlations between particular genotypes and changes in markers of cardiovascular status such as, would result, e.g., from particular treatment regimens. In this manner, it is possible to correlate genotypes with responsivity to particular treatments. One of ordinary skill in the art will recognize that a variety of different statistical tests can be used to establish a correlation.

In certain embodiments, two or more polymorphic variants form a haplotype. Such polymorphic variants may be variants disclosed herein, variants known in the art, or variants yet to be described. The invention encompasses determining whether an individual has a particular haplotype comprising one or more of the polymorphic variants disclosed herein. In some embodiments, a haplotype is associated with an increased risk of development or occurrence of a cardiovascular disease or event. Without wishing to be bound by any theory, such haplotypes may provide better predictive/diagnostic information than a single SNP.

In certain embodiments, a panel of SNPs and/or haplotypes is defined that provides diagnostic and/or prognostic information when an individual is genotyped with respect to the SNPs and/or haplotypes. In certain embodiments, a panel includes at least 2 SNPs, wherein the SNPs are substantially unlinked. For example, the recombination frequency between each pair of SNPs may be approximately 0.5. In certain embodiments, a panel includes at least 2, 3, 4, 5, 6, 7, 8, 9, 10, or more substantially unlinked SNPs. Of course a panel may also include one or more SNPs that are linked, e.g., in strong LD, with at least one other SNP. In some embodiments, at least 2 SNPs or haplotypes include a polymorphic variant that contributes a relative risk of at least 1.1 to an individual's overall risk of developing CVD and/or suffering a major coronary event within a defined period of time. In certain embodiments, at least 3, 4, 5, 6, 7, 8, 9, or 10 of the SNPs or haplotypes includes such a polymorphic variant. In certain embodiments, a panel includes at least 2 SNPs or haplotypes that are each present in at least 5% of a target population, each of which includes a polymorphic variant that contributes a relative risk of at least 1.1 to an individual's overall risk of developing CVD and/or suffering a major coronary event within a defined period of time. In certain embodiments of the invention at least 3, 4, 5, 6, 7, 8, 9, or 10 of the SNPs or haplotypes include such a polymorphic variant.

In certain embodiments, results obtained from the panel predict the risk for developing CVD and/or the risk of experiencing a major coronary event. In certain embodiments, subsequent identification and evaluation of an individual's haplotype can then help to guide specific and individualized therapy. The risk can be, e.g., absolute risk, which can be expressed in terms of the likelihood (e.g.,. % likelihood) that an individual will manifest a symptom or sign of CVD and/or will experience a major coronary event within a defined time period. Additionally or alternatively, the risk can be expressed in terms of relative risk, e.g., a factor that expressed the degree to which the individual is at increased risk relative to the risk the individual would face if his or her genotype with respect to one of more of the polymorphisms or haplotypes was different. Individuals can be stratified based on their risk. Such stratification can be used, for example, to select individuals who would be likely to benefit from particular therapeutic regimens and/or can be used to identify individuals for a clinical trial. It should be emphasized that the information provided by the methods of the present invention can be qualitative or quantitative and can be expressed using any convenient means. It can be based on the evaluation of one or both alleles of a single polymorphism in an individual, or can be based on the evaluation of multiple polymorphisms and/or haplotypes.

A predictive panel can be used for genotyping of one or more individuals on a platform that can genotype multiple SNPs at the same time (multiplexing). In certain embodiments, platforms are, e.g., gene chips (Affymetrix) or the Luminex LabMAP reader. See, e.g., (Armstrong, B., et al., Cytometry 40: 102-108, 2000; Cai, H., et al., Genomics 66: 135-143, 2000; and Chen, J., et al., Genome Research 10: 549-557, 2000) for description of Luminex assays and their application for purposes of SNP genotyping. Such assays typically involve populations of fluorescent beads, which are evaluated using flow cytometery. Of course these multiplexed assays are of use for genotyping individual SNPs in either a single individual or multiple individuals.

In certain embodiments, information obtained from detecting one or more polymorphic variants is used together with information obtained by evaluating the existence of classical risk factors in a patient to provide an assessment of risk that includes the contribution of genetic factors that may or may not play a role in classical risk factors such as lipid levels. In certain embodiments, an individual's classical risk factors are evaluated according to the PROCAM method (Assmann, supra) and an individual is genotyped with respect to one or more of the CVDA SNPs and/or haplotypes described herein. The genotyping results in a relative risk ratio that is used to modify the PROCAM score. For example, the PROCAM score may be multiplied by a relative risk determined based on the genotype of the individual, or a value is added to or subtracted from the PROCAM score to provide a modified PROCAM score. In certain embodiments, the invention therefore provides methods for determining whether an individual has or is susceptible to development or occurrence of a cardiovascular disease or event, wherein the individual is in need of such determination, the method comprising the step of: combining information derived from an assessment of one or more classical risk factors and/or cardiovascular status markers together with genetic information obtained by (a) detecting a polymorphic variant of a CVDA polymorphism in the individual; or (b) detecting a polymorphic variant in strong LD with a CVDA polymorphism; or (c) detecting a haplotype comprising a polymorphic variant of the CVDA polymorphism in the individual; or (d) detecting an allele of a gene comprising the polymorphic variant of the CVDA polymorphism in the individual. It will be appreciated that the combining can be performed in any of a wide variety of ways.

In certain embodiments, the invention provides a database or other suitably organized and optionally searchable compendium of information comprising a list or other suitable form of presentation of CVDA polymorphisms and/or polymorphic sequences, haplotypes, and/or linked polymorphisms, stored on a computer-readable medium, wherein the contents of the database may be largely or entirely limited to polymorphisms that have been identified as useful in performing genotyping for assessing an individual's susceptibility to CVD, etc., as described herein. The database is distinguished from general compendia of information regarding polymorphisms, such as those described above, in that it specifically groups, selects, and/or identifies the polymorphisms as being related to CVD. In some embodiments the database includes information such as a relative risk, allele frequencies, or the like. It will be appreciated that the information can be stored in any of a wide variety of formats. The database may include results of genotyping one or more individuals with respect to one or more of the CVDA polymorphisms and/or haplotypes described herein. The results can be results of one or more of the tests described herein. The invention also encompasses a method comprising the step of electronically sending or receiving information such as that present in a database of the invention and/or electronically sending or receiving results of a genotyping test as described herein.

Isolated Polymorphic Nucleic Acids, Probes, and Vectors

The present invention provides isolated nucleic acids comprising the polymorphic positions and specific polymorphic variants described herein for human genes; vectors comprising the nucleic acids, and transformed host cells comprising the vectors. The invention also provides probes, primers, and other reagents that are useful for detecting the polymorphic variants of such polymorphisms.

In practicing the present invention, many conventional techniques in molecular biology, microbiology, and recombinant DNA technology may be used. Such techniques are well known and are explained fully in, for example, Sambrook et al., supra, Ausubel, F. (ed.), supra, DNA Cloning: A Practical Approach, Volumes I and II, 1985 (D. N. Glover ed.); Oligonucleotide Synthesis, 1984, (M. L. Gait ed.); Nucleic Acid Hybridization, 1985, (Hames and Higgins); Ausubel et al., Current Protocols in Molecular Biology, 1997, (John Wiley and Sons); and Methods in Enzymology Vol. 154 and Vol. 155 (Wu and Grossman, and Wu, eds., respectively).

Since the human genome has been sequenced and is publicly available, e.g., at the NCBI website, knowing the identity of the SNPs listed in Tables 1 and 2 provides the artisan with the genomic DNA sequence located adjacent to, e.g., upstream and/or downstream of the SNP. A portion of the surrounding sequence for each SNP is provided in Tables 1 and 2. A sequence of any particular length can be selected. In certain embodiments, the portion surrounding the SNP can be used as a probe to identify a longer genomic sequence of a cDNA. Thus, in certain embodiments, the invention provides a cDNA comprising any of the polymorphic sites identified herein.

In certain embodiments, the invention provides an isolated nucleic acid comprising or immediately adjacent to the position of a SNP identified in Tables 1 and 2. The isolated nucleic acid can be of any desired length. Insertion of nucleic acids (typically DNAs) comprising sequences encompassed by the present invention into a nucleic acid vector is easily accomplished when the termini of both the nucleic acids and the vector comprise compatible ends, such as those generated by cleavage with a restriction enzyme. If this is not possible, the termini of the DNAs and/or vector can be modified by digesting back single-stranded DNA overhangs generated by restriction endonuclease cleavage to produce blunt ends, or to achieve the same result by filling in the single-stranded termini with an appropriate DNA polymerase. In certain embodiments, a specific sequence at the ends of the nucleic acids to be inserted, the vectors, or both may be produced, e.g., by ligating nucleotide sequences (linkers) onto the termini. Such linkers may comprise specific oligonucleotide sequences that define desired restriction sites. Restriction sites can also be generated by the use of the polymerase chain reaction (PCR). See, e.g., Saiki et al., Science 239:48, 1988. The cleaved vector and the DNA fragments may also be modified if required by homopolymeric tailing. One of ordinary skill in the art will appreciate that cloning and manipulation of nucleic acids is wholly routine in the art, and kits are commercially available for performing virtually any desired manipulation.

The nucleic acids may be isolated directly from cells or may be chemically synthesized using known methods. Alternatively, the polymerase chain reaction (PCR) method can be used to produce inventive nucleic acids, using either chemically synthesized strands or genomic material as templates. Primers used for PCR can be synthesized using the sequence information provided herein and can further be designed to introduce appropriate new restriction sites, if desirable, to facilitate incorporation into a given vector for recombinant expression.

Reagents and Methods for Modulating Expression and/or Activity of CVDA Polynucleotides and Polypeptides

The CVDA genes and their encoded mRNA and polypeptides are potential therapeutic targets for cardiovascular disease. Therefore, it is desirable to be able to modulate their expression and/or activity, both for therapeutic and other purposes. In certain embodiments, the invention therefore provides a variety of methods for altering expression and/or functional activity of a CVDA gene, which are further described below. The invention encompasses methods for screening compounds for preventing or treating a cardiovascular disease by assaying the ability of the compounds to modulate the expression of one or more of the CVDA genes disclosed herein or activity of the protein products of these genes. Appropriate screening methods include, but are not limited to, assays for identifying compounds and other substances that interact with (e.g., bind to) the target gene or protein.

In certain embodiments, the invention provides an antisense nucleic acid that inhibits expression of a CVDA gene. In certain embodiments, such an antisense nucleic acid selectively inhibits a CVDA polymorphic variant of a CVDA gene. As is known in the art, antisense nucleic acids are generally single-stranded nucleic acids (DNA, RNA, modified DNA, modified RNA, or peptide nucleic acids) complementary to a portion of a target nucleic acid (e.g., an mRNA transcript) and therefore able to bind to the target to form a duplex. Typically they are oligonucleotides that range from 15 to 35 nucleotides in length but may range from 10 up to approximately 50 nucleotides in length. Binding typically reduces or inhibits the function of the target nucleic acid. For example, antisense oligonucleotides may block transcription when bound to genomic DNA, inhibit translation when bound to mRNA, and/or lead to degradation of the nucleic acid. Reduction in expression of a CVDA polypeptide may be achieved by the administration of an antisense nucleic acid or peptide nucleic acid (PNA) comprising sequences complementary to those of the mRNA that encodes the polypeptide. Antisense technology and its applications are well known in the art and are described in Phillips, M. I. (ed.) Antisense Technology, Methods Enzymol., Volumes 313 and 314, Academic Press, San Diego, 2000, and references mentioned therein. See also Crooke, S. (ed.) “Antisense Drug Technology: Principles, Strategies, and Applications” (1^{st} ed), Marcel Dekker; ISBN: 0824705661; 1st edition (2001) and references therein.

Peptide nucleic acids (PNA) are analogs of DNA in which the backbone is a pseudopeptide rather than a sugar. PNAs mimic the behavior of DNA and bind to complementary nucleic acid strands. The neutral backbone of a PNA can result in stronger binding and greater specificity than normally achieved using DNA or RNA. Binding typically reduces or inhibits the function of the target nucleic acid. Peptide nucleic acids and their use are described in Nielsen, P. E. and Egholm, M., (eds.) “Peptide Nucleic Acids: Protocols and Applications” (First Edition), Horizon Scientific Press, 1999.

According to certain embodiments, the antisense oligonucleotides have any of a variety of lengths. For example, such antisense oligonucleotides may comprise between 8 and 60 contiguous nucleotides complementary to an mRNA encoded by a CVDA gene, between 10 and 60 contiguous nucleotides complementary to an mRNA encoded by a CVDA gene, or between 12 and 60 contiguous nucleotides complementary to an mRNA encoded by a CVDA gene. According to certain embodiments, the antisense oligonucleotide need not be perfectly complementary to the mRNA to which it hybridizes but may have, for example, up to 1 or 2 mismatches per 10 nucleotides.

The invention further encompasses a method of inhibiting expression of a CVDA gene in a cell or individual comprising delivering an antisense oligonucleotide to the cell or individual or expressing such an antisense oligonucleotide within a cell or cells of the individual. Additionally or alternatively, inventive methods include treating or preventing a cardiovascular disease or condition comprising steps of (i) providing a individual in need of treatment for or prevention of a cardiovascular disease or condition; and (ii) administering a pharmaceutical composition comprising an effective amount of a an antisense oligonucleotide to the individual, wherein the antisense oligonucleotide inhibits expression of a CVDA gene.

In certain embodiments, the invention provides a ribozyme designed to cleave an mRNA encoded by a CVDA gene. Ribozymes (catalytic RNA molecules that are capable of cleaving other RNA molecules) represent another approach to reducing gene expression. Ribozymes can be designed to cleave specific mRNAs corresponding to a gene of interest. Their use is described in U.S. Pat. No. 5,972,621, and references therein. Extensive discussion of ribozyme technology and its uses is found in Rossi, J. J., and Duarte, L. C., Intracellular Ribozyme Applications: Principles and Protocols, Horizon Scientific Press, 1999.

The invention further encompasses methods of inhibiting expression of a CVDA polypeptide in a cell or individual comprising delivering a ribozyme designed to cleave an mRNA encoded by a CVDA gene to the cell or individual or expressing such a ribozyme within a cell or cells of the individual. Additionally or alternatively, the invention provides methods of treating or preventing a cardiovascular disease or condition comprising steps of (i) providing a individual in need of treatment for a cardiovascular disease or condition; and (ii) administering a pharmaceutical composition comprising an effective amount of a ribozyme designed to cleave an mRNA encoded by a CVDA gene to the individual, thereby alleviating the condition.

RNA interference (RNAi) is a mechanism of post-transcriptional gene silencing mediated by double-stranded RNA (dsRNA), which is distinct from the antisense and ribozyme-based approaches described above. dsRNA molecules are believed to direct sequence-specific degradation of mRNA that contain regions complementary to one strand (the antisense strand) of the dsRNA in cells of various types after first undergoing processing by an RNase III-like enzyme called DICER (Bernstein et al., Nature 409:363, 2001) into smaller dsRNA molecules. Such molecules comprise two 21 nt strands, each of which has a 5′ phosphate group and a 3′ hydroxyl, and includes a 19 nt region precisely complementary with the other strand, so that there is a 19 nt duplex region flanked by 2 nt-3′ overhangs and are known as short interfering RNA (siRNA). An siRNA typically comprises a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

RNAi can also be achieved using molecules referred to as short hairpin RNAs (shRNA), which are single RNA molecules comprising at least two complementary portions capable of self-hybridizing to form a duplex structure sufficiently long to mediate RNAi (typically at least 19 base pairs in length), and a loop, typically between approximately 1 and 10 nucleotides in length and more commonly between 4 and 8 nucleotides in length that connects the two nucleotides that form the last nucleotide pair at one end of the duplex structure. shRNAs are thought to be processed into siRNAs by the conserved cellular RNAi machinery. Thus shRNAs are precursors of siRNAs and are similarly capable of inhibiting expression of a target transcript.

siRNAs and shRNAs have been shown to downregulate gene expression when transferred into mammalian cells by such methods as transfection, electroporation, or microinjection, or when expressed in cells via any of a variety of plasmid-based approaches. RNA interference using siRNA and/or shRNA is reviewed in, e.g., Tuschl, T., Nat. Biotechnol., 20: 446-448, May 2002. See also Yu, J., et al., Proc. Natl. Acad. Sci., 99(9), 6047-6052, 2002; Sui, G., et al., Proc. Natl. Acad. Sci., 99(8), 5515-5520, 2002; Paddison, P., et al., Genes and Dev., 16, 948-958, 2002; Brummelkamp, T., et al., Science, 296, 550-553, 2002; Miyagashi, M. and Taira, K., Nat. Biotech., 20, 497-500, 2002; Paul, C., et al., Nat. Biotech., 20, 505-508, 2002. A number of variations in structure, length, number of mismatches, size of loop, identity of nucleotides in overhangs, etc., are consistent with effective RNAi-mediated gene silencing. For example, one or more mismatches between the target mRNA and the complementary portion of the siRNA or shRNA may still be compatible with effective silencing.

It is thought that intracellular processing (e.g., by DICER) of a variety of different precursors results in production of RNAs of various kinds that are capable of effectively mediating gene silencing. For example, in addition to the siRNA and shRNA structures described above, DICER can process ˜70 nucleotide hairpin precursors with imperfect duplex structures, i.e., duplexes that are interrupted by one or more mismatches, bulges, or inner loops within the stem of the hairpin into single-stranded RNAs called microRNAs (miRNA) that are believed to hybridize within the 3′ UTR of a target mRNA and repress translation. See, e.g., Lagos-Quintana, M. et al., Science, 294, 853-858, 2001; Pasquinelli, A., Trends in Genetics, 18(4), 171-173, 2002, and references in the foregoing two articles for discussion of miRNAs and their mechanisms of silencing.

Accordingly, In certain embodiments, the invention provides siRNAs and shRNAs that inhibit expression of an mRNA encoded by any of the CVDA genes disclosed herein. An RNAi agent is considered to inhibit expression of a target transcript and thus to be targeted to the transcript if the stability or translation of the target transcript is reduced in the presence of the siRNA as compared with its absence. Typically the antisense portion of an RNAi agent shows at least about 80%, at least about 90%, at least about 91%, 92%, 93%, 94%, 95%, 96%, 97%, 98%, 99%, or 100% precise sequence complementarity with the target transcript for a stretch of at least about 17, 18 or 19 to about 21-23 or more nucleotides.

The invention encompasses methods of inhibiting expression of a CVDA gene in a cell or individual comprising delivering an siRNA or shRNA targeted to an mRNA encoded by a CVDA gene to the cell or individual. Additionally or alternatively, the invention provides methods of treating or preventing a cardiovascular disease or condition comprising steps of (i) providing a individual in need of treatment for atherosclerosis or a disease or condition associated with atherosclerosis; and (ii) administering a pharmaceutical composition comprising an effective amount of an siRNA or shRNA targeted to an mRNA encoded by a CVDA gene to the individual, thereby alleviating the condition.

As mentioned above, siRNAs and shRNAs have been shown to effectively reduce gene expression when expressed intracellularly, e.g., by delivering vectors such as plasmids, viral vectors such as adenoviral, retroviral or lentiviral vectors, or viruses to cells. Such vectors, referred to herein as RNAi-inducing vectors, are vectors whose presence within a cell results in transcription of one or more RNAs that self-hybridize or hybridize to each other to form an shRNA or siRNA. In general, the vector comprises a nucleic acid operably linked to expression signal(s) so that one or more RNA molecules that hybridize or self-hybridize to form an siRNA or shRNA are transcribed when the vector is present within a cell. Thus the vector provides a template for intracellular synthesis of the RNA or RNAs or precursors thereof. The vector will thus contain a sequence or sequences whose transcription results in synthesis of two complementary RNA strands having the properties of siRNA strands described above, or a sequence whose transcription results in synthesis of a single RNA molecule containing two complementary portions separated by an intervening portion that forms a loop when the two complementary portions hybridize to one another.

Selection of appropriate siRNA and shRNA sequences can be performed according to guidelines well known in the art, e.g., taking factors such as desirable GC content into consideration. See, e.g., (Dykxhoorn, D. M., et al., Nature Reviews Molecular Cell Biology 4: 457-467, 2003; Elbashir, S. M., et al., Nature. 411:494-498, 2001; Elbashir, S. M., et al., Genes and Development, 15: 188-200, 2001; and WO 01/75164 and U.S. Pub. Nos. 20020086356 and 20030108923 for further information.

As is known in the art, by selecting about 5 siRNAs it will almost always be possible to identify an effective sequence. If necessary additional siRNAs can be designed and tested. It may be desirable to perform a systematic screen to identify highly effective siRNAs. A number of computer programs that aid in the selection of effective siRNA/shRNA sequences are known in the art, which yield even higher percentages of effective siRNAs. See, e.g., Cui, W., et al., “OptiRNai, a Web-based Program to Select siRNA Sequences”, Proceedings of the IEEE Computer Society Conference on Bioinformatics, p. 433, 2003. Algorithms for selecting effective siRNA are also described in Reynolds, A., et al., Nat Biotechnol., 22(3):326-30, 2004. Pre-designed siRNAs targeting over 95% of the mouse or human genome are commercially available, e.g., from Ambion and/or Cenix Biosciences. As is known in the art, siRNAs and shRNAs can be delivered using a variety of delivery agents that increase their cellular uptake or potency or protect them from degradation.

Antisense nucleic acids, ribozymes, siRNAs, or shRNAs can be delivered to cells by standard techniques such as microinjection, electroporation, or transfection. Antisense nucleic acids, ribozymes, siRNAs, or shRNAs can be formulated as pharmaceutical compositions and delivered to an individual using a variety of approaches. According to certain embodiments, the delivery of antisense, ribozyme, siRNA, or shRNA molecules is accomplished via a gene therapy approach in which vectors (e.g., viral vectors such as retroviral, lentiviral, or adenoviral vectors, etc.) are delivered to a cell or individual, or cells directing expression of the molecules (e.g., cells into which a vector directing expression of the molecule has been introduced) are administered to the individual.

It may be advantageous to employ various nucleotide modifications and nucleotide analogs to confer desirable properties on the antisense nucleic acid, ribozyme, siRNA, or shRNA. Numerous nucleotide analogs, nucleotide modifications, and modifications elsewhere in a nucleic acid chain are known in the art, and their effect on properties such as hybridization and nuclease resistance has been explored. For example, various modifications to the base, sugar and internucleoside linkage have been introduced into oligonucleotides at selected positions, and the resultant effect relative to the unmodified oligonucleotide compared. A number of modifications have been shown to alter one or more aspects of the oligonucleotide such as its ability to hybridize to a complementary nucleic acid, its stability, etc. For example, useful 2′-modifications include halo, alkoxy and allyloxy groups. U.S. Pat. Nos. 6,403,779, 6,399,754, 6,225,460, 6,127,533, 6,031,086, 6,005,087, 5,977,089, and references therein disclose a wide variety of nucleotide analogs and modifications that may be of use in the practice of the present invention. See also Crooke, S. (ed.), referenced above, and references therein. As will be appreciated by one of ordinary skill in the art, analogs and modifications may be tested using, e.g., the assays described herein or other appropriate assays, in order to select those that effectively reduce expression of the target nucleic acid. The analog or modification advantageously results in a nucleic acid with increased absorbability (e.g., increased absorbability across a mucus layer, increased oral absorption, etc.), increased stability in the blood stream or within cells, increased ability to cross cell membranes, etc.

Antisense RNAs, ribozymes, siRNAs or shRNAs may be prepared by any method known in the art for the synthesis of nucleic acid molecules. These include, but are not limited to, techniques for chemical synthesis such as solid phase phosphoramidite chemical synthesis. In the case of siRNAs, the structure may be stabilized, for example by including nucleotide analogs at one or more free strand ends in order to reduce digestion, e.g., by exonucleases. This may also be accomplished by the use of deoxy residues at the ends, e.g., by employing dTdT overhangs at each 3′ end. Alternatively, antisense, ribozyme, siRNA or shRNA molecules may be generated by in vitro transcription of DNA sequences encoding the relevant molecule. Such DNA sequences may be incorporated into a wide variety of vectors with suitable RNA polymerase promoters such as T7, T3, or SP6.

Antisense, ribozyme, siRNA or shRNA molecules may be generated by intracellular synthesis of small RNA molecules, as described above, which may be followed by intracellular processing events. For example, intracellular transcription may be achieved by cloning templates into RNA polymerase III transcription units, e.g., under control of a U6 or H1 promoter. In one approach for intracellular synthesis of siRNA, sense and antisense strands are transcribed from individual promoters, which may be on the same construct. The promoters may be in opposite orientation so that they drive transcription from a single template, or they may direct synthesis from different templates. However, it may be advantageous to express a single RNA molecule that self-hybridizes to form a hairpin RNA that is then cleaved by DICER within the cell.

In some embodiments, an antisense oligonucleotide, RNAi agent, or ribozyme specifically inhibits expression of an allele of the CVDA gene that is associated with cardiovascular disease, e.g., the allele comprises one or more of the CVDA polymorphic variants disclosed herein. For example, an antisense oligonucleotide or antisense strand of an siRNA may be complementary to the disease-associated polymorphic variant at the polymorphic position. In some embodiments, an antisense oligonucleotide, ribozyme, or RNAi agent does not appreciably inhibit the polymorphic variant that is not associated with cardiovascular disease. By “does not appreciably inhibit” is meant that the expression level in the presence of the inhibitory agent is at least 90% of the expression level in the absence of the agent. In certain embodiments the expression level of the polymorphic variant that is not associated with cardiovascular disease is inhibited by less than 50% by the antisense oligonucleotide, ribozyme, or RNAi agent.

Antisense, ribozyme, siRNA, or shRNA molecules for use in accordance with the present invention may be introduced into cells by any of a variety of methods. For instance, antisense, ribozyme, siRNA, or shRNA molecules or vectors encoding them can be introduced into cells via conventional transformation or transfection techniques. As used herein, the terms “transformation” and “transfection” are intended to refer to a variety of art-recognized techniques for introducing foreign nucleic acid (e.g., DNA or RNA) into a host cell, including calcium phosphate or calcium chloride co-precipitation, DEAE-dextran-mediated transfection, lipofection, injection, or electroporation.

Vectors that direct in vivo synthesis of antisense, ribozyme, siRNA, or shRNA molecules constitutively or inducibly can be introduced into cell lines, cells, or tissues. In certain embodiments, inventive vectors are gene therapy vectors (e.g., adenoviral vectors, adeno-associated viral vectors, retroviral or lentiviral vectors, or various nonviral gene therapy vectors) appropriate for the delivery of a construct directing transcription of an siRNA to mammalian cells, including, but not limited to, human cells.

In certain embodiments, siRNA, shRNA, antisense, or ribozyme compositions reduce the level of a target transcript and/or its encoded protein by at least 2-fold. In certain embodiments, siRNA, shRNA, antisense, or ribozyme compositions reduce the level of a target transcript and/or its encoded protein by at least 4-fold. In certain embodiments, siRNA, shRNA, antisense, or ribozyme compositions reduce the level of a target transcript and/or its encoded protein by at least 10-fold or more. The ability of a candidate siRNA to reduce expression of the target transcript and/or its encoded protein may readily be tested using methods well known in the art including, but not limited to, Northern blots, RT-PCR, microarray analysis in the case of the transcript, and various immunological methods such as Western blot, ELISA, immunofluorescence, etc., in the case of the encoded protein. In addition, the potential of any siRNA, shRNA, antisense, or ribozyme composition for treatment of a particular condition or disease associated with atherosclerosis may also be tested in appropriate animal models or in human individuals, as is the case for all methods of treatment described herein. Appropriate animal models include, but are not limited to, mice, rats, rabbits, sheep, dogs, etc., with experimentally induced atherosclerosis.

Nucleic acids described herein may be delivered to an individual using any of a variety of approaches, including those applicable to non-nucleic acid agents such as IV, intranasal, oral, etc. According to certain embodiments, such nucleic acids are delivered via a gene therapy approach, in which a construct capable of directing expression of one or more of the inventive nucleic acids is delivered to cells or to the individual (ultimately to enter cells, where transcription may occur).

Additional methods for identifying compounds capable of modulating gene expression are described, for example, in U.S. Pat. No. 5,976,793. These methods may be used either to identify compounds that increase gene expression or to identify compounds that decrease gene expression. Additional methods for identifying agents that increase expression of genes are found in Ho, S., et al., Nature, 382, pp. 822-826, 1996, which describes homodimeric and heterodimeric synthetic ligands that allow ligand-dependent association and disassociation of a transcriptional activation domain with a target promoter to increase expression of an operatively linked gene.

Expression can also be increased by introducing additional copies of a coding sequence into a cell of interest, e.g., by introducing a nucleic acid comprising the coding sequence into the cell. In certain embodiments, a coding sequence is operably linked to regulatory elements such as promoters, enhancers, etc., that direct expression of the coding sequence in the cell. A nucleic acid may comprise a complete CVDA gene, or a portion thereof that contains the coding region of the gene. A nucleic acid may be introduced into cells grown in culture or cells in an individual using any suitable method, e.g., any of those described above.

Polymorphic Polypeptides and Polymorphism-Specific Binding Agents

In certain embodiments, the present invention provides isolated peptides and polypeptides encoded by genes listed in Tables 1 and 2, wherein the genes comprise one or more polymorphic positions disclosed herein. In certain embodiments, such peptides and polypeptides are useful in screening targets to identify drugs for the treatment and/or prevention of cardiovascular disease. In certain embodiments, such peptides and polypeptides are capable of eliciting antibodies in a suitable host animal that react specifically with a polypeptide comprising the polymorphic position and distinguish it from other polypeptides having a different sequence at that position. In certain embodiments, a peptide or polypeptide is used to identify a specific binding reagent that binds to the peptide or polypeptide. In certain aspects, the invention thus provides antibodies and other reagents that specifically bind to a polypeptide having a specific amino acid at a polymorphic position. Certain inventive antibodies possess high affinity, e.g., a K_{d} of <200 nM, <100 nM, or lower for their target.

Certain inventive polypeptides are at least five or more residues in length. In certain embodiments, polypeptides of the present invention are at least fifteen residues. Certain methods for obtaining these polypeptides are described below. A variety of conventional techniques in protein biochemistry and immunology are known and my be used in accordance with the present invention. For example, certain conventional techniques are explained in Immunochemical Methods in Cell and Molecular Biology, 1987 (Mayer and Waler, eds; Academic Press, London); Scopes, 1987, Protein Purification: Principles and Practice, Second Edition (Springer-Verlag, N.Y.), Handbook of Experimental Immunology, 1986, Volumes I-IV (Weir and Blackwell eds.), Harlow, supra, and other references listed above.

Nucleic acids comprising protein-coding sequences can be used to direct recombinant expression of polypeptides encoded by genes disclosed herein in intact cells or in cell-free translation systems. The nucleic acids can be isolated from cells, nucleic acid libraries (e.g., cDNA libraries), synthesized chemically, etc. The genetic code can be used to design polynucleotides encoding the desired amino acid sequences. If desired, the sequence of the polynucleotides can be modified to optimize expression in a host cell or organism of choice. The polypeptides may be isolated from human cells, or from heterologous organisms or cells (including, but not limited to, bacteria, fungi, insect, plant, and mammalian cells) into which an appropriate protein-coding sequence has been introduced and expressed. Furthermore, the polypeptides may be part of recombinant fusion proteins.

Peptides and polypeptides may be chemically synthesized by commercially available automated procedures, including, without limitation, exclusive solid phase synthesis, partial solid phase methods, fragment condensation or classical solution synthesis. In certain embodiments, polypeptides are advantageously prepared by solid phase peptide synthesis as described by Merrifield, J. Am. Chem. Soc. 85:2149, 1963.

Methods for polypeptide purification are well-known in the art, including, without limitation, preparative disc-gel electrophoresis, isoelectric focusing, HPLC, reversed-phase HPLC, gel filtration, ion exchange and partition chromatography, and countercurrent distribution. For some purposes, it may be advantageous to produce the polypeptide in a recombinant system in which the protein contains an additional sequence tag that facilitates purification, such as, for example, a polyhistidine sequence. The polypeptide can then be purified from a crude lysate of the host cell by chromatography on an appropriate solid-phase matrix. Additionally or alternatively, antibodies produced against peptides encoded by genes disclosed herein, can be used as purification reagents. Other purification methods that may be used will be known to those of ordinary skill in the art.

The present invention also encompasses derivatives and homologues of the polypeptides. For some purposes, nucleic acid sequences encoding the peptides may be altered by substitutions, additions, or deletions that provide for functionally equivalent molecules, e.g., function-conservative variants. For example, one or more amino acid residues within the sequence can be substituted by another amino acid of similar properties, such as, for example, positively charged amino acids (arginine, lysine, and histidine); negatively charged amino acids (aspartate and glutamate); polar neutral amino acids; and non-polar amino acids. In certain embodiments, a derivative or homologue of a polypeptide is at least 80% identical, at least 90% identical, at least 95%, or at least 99% identical to the polypeptide. In certain embodiments a derivative or homologue of a polypeptide has 5, 10, 20, 30, 40, or 50 or more amino acid deletions, additions, or substitutions relative to the polypeptide.

Polypeptides may be modified by, for example, phosphorylation, sulfation, acylation, or other protein modifications. They may also be modified with a label capable of providing a detectable signal, either directly or indirectly, including, but not limited to, radioisotopes and fluorescent compounds. The polypeptides may include a tag, e.g., an epitope tag such as an HA tag, 6× His tag, GST tag, etc., which may be useful for detection and/or purification of the polypeptide.

The present invention also encompasses antibodies that specifically recognize polypeptides differing at one or more polymorphic positions and that distinguish a peptide or polypeptide containing a particular polymorphic variant from one that contains a different sequence at that position. Such polymorphic position-specific antibodies include, for example, polyclonal and monoclonal antibodies. Such antibodies may be generated in an animal host by immunization with polypeptides encoded by genes disclosed herein or may be identified using methods such as phage display. The immunogenic components used to generate antibodies may be isolated from human cells or produced in recombinant systems. Such antibodies may also be produced in recombinant systems programmed with appropriate antibody-encoding DNA. Additionally or alternatively, antibodies may be constructed by biochemical reconstitution of purified heavy and light chains. Such antibodies include hybrid antibodies (e.g., containing two sets of heavy chain/light chain combinations, each of which recognizes a different antigen), chimeric antibodies (i.e., in which either the heavy chains, light chains, or both, are fusion proteins), and univalent antibodies (e.g., comprised of a heavy chain/light chain complex bound to the constant region of a second heavy chain). Also encompassed are Fab fragments, including Fab′ and F(ab)₂ fragments of antibodies. Methods for the production of all of the above types of antibodies and derivatives are well-known in the art and are discussed in more detail below. For example, techniques for producing and processing polyclonal antisera are disclosed in Mayer and Walker, Immunochemical Methods in Cell and Molecular Biology, 1987 (Academic Press, London). The general methodology for making monoclonal antibodies by hybridomas is well known. See, e.g., Schreier et al., Hybridoma Techniques, 1980; U.S. Pat. Nos. 4,341,761; 4,399,121; 4,427,783; 4,444,887; 4,466,917; 4,472,500; 4,491,632; and 4,493,890. Panels of monoclonal antibodies produced against peptides encoded by genes disclosed herein can be screened for various properties, including, for example, isotype, epitope affinity, etc.

Antibodies of the present invention can be purified by standard methods, including but not limited to preparative disc-gel electrophoresis, isoelectric focusing, HPLC, reversed-phase HPLC, gel filtration, ion exchange and partition chromatography, and countercurrent distribution. Purification methods for antibodies are disclosed, e.g., in The Art of Antibody Purification, Amicon Division, W. R. Grace & Co, 1989. General protein purification methods are described in Protein Purification: Principles and Practice, R. K. Scopes, Ed., 1987, Springer-Verlag, New York, N.Y.

Methods for determining the immunogenic capability of the disclosed sequences and the characteristics of the resulting sequence-specific antibodies and immune cells are well-known in the art. For example, antibodies elicited in response to a peptide comprising a particular polymorphic sequence can be tested for their ability to specifically recognize that polymorphic sequence, e.g., to bind differentially to a peptide or polypeptide comprising the polymorphic sequence and thus distinguish it from a similar peptide or polypeptide containing a different sequence at the same position.

A variety of engineered ligand-binding proteins with antibody-like specific binding properties are known in the art. For example, anticalins offer an alternative type of ligand-binding protein, which is constructed on the basis of lipocalins as a scaffold (Skerra, J., J. Biotechnol., 74(4):257-75, 2001). Affibodies, which are binding proteins generated by phage display from combinatorial libraries constructed using the protein A-derived Z domain as a scaffold, can also be used. See, e.g., Nord K, Eur J Biochem., 268(15):4269-77, 2001. Thus the invention provides, for example, an affibody or anticalin that specifically binds to a CVDA polypeptide.

In certain embodiments, the invention also provides a variety of different additional specific binding agents which may be, for example, peptides, non-immunoglobulin polypeptides, nucleic acids, protein nucleic acids (PNAs), aptamers, small molecules, etc. Such agents will be collectively referred to herein as “ligands”. Ligands that specifically bind to any of the polymorphic forms of the CVDA polypeptides described herein may be identified using any of a variety of approaches. For example, ligands may be identified by screening libraries, e.g., small molecule libraries. Naturally occurring or artificial (non-naturally occurring) ligands, particularly peptides or polypeptides, may be identified using a variety of approaches including, but not limited to, those known generically as two- or three-hybrid screens, the first version of which was described in Fields S. and Song O., Nature, 340(6230):245-6, 1989. Nucleic acid or modified nucleic acid ligands may be identified using, e.g., systematic evolution of ligands by exponential enrichment (SELEX) (Tuerk, C. and Gold., L, Science 249(4968): 505-10, 1990), or any of a variety of directed evolution techniques that are known in the art. For example, an aptamer is an oligonucleotide (e.g., DNA, RNA, which can include various modified nucleotides, e.g., 2′-O-methyl modified nucleotides) that binds to a particular protein. See, e.g., Brody E N, Gold L. J Biotechnol., 74(1):5-13, 2000. In certain embodiments, the ligand is an aptamer that binds to a CVDA polypeptide or polymorphic form thereof. Screens using nucleic acids, peptides, or polypeptides as candidate ligands may utilize nucleic acids, peptides, or polypeptides that incorporate any of a variety of nucleotide analogs, amino acid analogs, etc. Various nucleotide analogs are known in the art, and other modifications of a nucleic acid chain, e.g., in the backbone, can also be used. Peptide or polypeptide ligands may comprise amino acids that do not occur naturally (e.g., that are not used by organisms in naturally-occurring polypeptide sequences).

Antibody-Based Diagnostic Methods

In certain embodiments, the invention provides methods for detecting CVDA polymorphic variants, haplotypes, and/or alleles in a biological sample that employ a specific binding reagent such as an antibody. In certain embodiments, inventive methods comprises steps of: (i) contacting a sample with one or more antibodies, wherein each of the antibodies is specific for a particular polymorphic form of a protein encoded by a gene disclosed herein, under conditions in which a stable antigen-antibody complex can form between the antibody and antigenic components in the sample; and (ii) detecting any antigen-antibody complex formed in step (i) using any suitable means known in the art, wherein the detection of a complex indicates the presence of the particular polymorphic form in the sample.

Typically, immunoassays use either a labeled antibody or a labeled antigenic component (e.g., that competes with the antigen in the sample for binding to the antibody). Suitable labels include without limitation enzyme-based, fluorescent, chemiluminescent, radioactive, colorimetric, or dye molecules. Assays that amplify the signals from the probe are also known, such as, for example, those that utilize biotin and avidin, and enzyme-labeled immunoassays, such as ELISA assays.

It will be appreciated that methods described herein can be practiced using other reagents that exhibit specific binding to a polymorphic form of a polymorphic polypeptide.

Kits

In certain embodiments, inventive methods include methods for detecting the polymorphic variants, haplotypes, and alleles described herein. In certain embodiments, the invention provides methods for determining the identity of the polymorphic variants of polymorphic regions present in the genes disclosed herein, wherein specific polymorphic variants are associated with cardiovascular disease. In certain embodiments, the invention provides a variety of different kits that can be used for carrying out certain of the inventive methods. In certain embodiments, kits can be used to determine whether an individual has or is at risk of developing a cardiovascular disease. Such information can be used, optionally together with information regarding classical cardiovascular risk factors, to provide an absolute or relative risk that the individual will suffer a major coronary event. In certain embodiments, such information is used to guide the selection of a therapeutic regimen for such individuals, e.g., to optimize their treatment.

In certain embodiments, a kit comprises a probe or primer which is capable of hybridizing to a nucleic acid and that can be used to determine whether the nucleic acid contains a polymorphic variant of a polymorphic region that is associated with a risk for cardiovascular disease. Such a kit may further comprise instructions for performing the assay and/or instructions for using the results for diagnosis of an individual as having, or being susceptible to, developing a cardiovascular disease, or for prognosis. For example, a kit may comprise an informational sheet or the like that describes how to interpret the results of the test and/or how to utilize the results of the test together with information regarding the existence or value of one or more classical risk factors in the individual, or together with a comprehensive classical risk factor profile of the individual.

Probes or primers of inventive kits can be any of the probes or primers described herein, e.g., a labeled primer or labeled probe, or collection of labeled primers or labeled probes. In certain embodiments, a kit comprises probes and/or primers suitable for detection of a plurality of CVDA polymorphic variants and/or CVDA haplotypes, or CVDA alleles of a gene containing one or more such variants. In certain embodiments, a kit comprises 1, 2, 3, 4, 5, 6, 7, 8, 9, 10, 15, 20, 25, 30, 35, 40, 45, 50, 60, 70, 80, 90, 100, 200, 300, 400, 500 or more probes or primers.

In certain embodiments, probes are covalently or noncovalently attached to a support such as a microparticle. In certain embodiments, probes are covalently or noncovalently attached to a substantially planar, rigid substrate or support. In certain embodiments, such a substrate is transparent to radiation of the excitation and emission wavelengths used for excitation and detection of typical labels (e.g., fluorescent labels, quantum dots, plasmon resonant particles, nanoclusters), e.g., between approximately 400-900 nm. Materials such as glass, plastic, quartz, etc., are suitable. For example, a glass slide or the like can be used.

Kits of the present invention may further include components for detecting polymorphic forms of proteins encoded by genes that comprise the polymorphic variants described herein, wherein the polymorphic variant is in a coding region of a gene and results in a change in the amino acid sequence of the encoded polypeptide. Such kits may include one or more polymorphism-specific antibodies or other reagents exhibiting specific binding such as affibodies, aptamers, etc. Such antibodies may be pre-labeled, e.g., with an enzyme or a detectable moiety. In some embodiments, an antibody may be unlabelled and ingredients for labeling may be included in the kit in separate containers, or a secondary, labeled antibody is provided. An antibody may be covalently or noncovalently attached to a microparticle or to a support or substrate, e.g., a substantially planar, rigid support or substrate. Kits may also contain other suitably packaged reagents and materials needed for the particular immunoassay protocol, including solid-phase matrices, if applicable, and standards, e.g., molecular weight standards.

In certain embodiments, inventive kits are adaptable to high-throughput and/or automated operation. For example, kits may be suitable for performing assays in multiwell plates and may utilize automated fluid handling and/or robotic systems, plate readers, etc. In some embodiments, flow cytometry is used.

One of ordinary skill in the art will appreciate that a number of other polymorphic variants, haplotypes, and/or alleles associated with cardiovascular disease are known in the art in addition to those described herein. In certain embodiments, one or more of such known polymorphic variants, haplotypes, and/or alleles is detected in addition to detecting one or more of the CVDA polymorphic variants, haplotypes, or alleles described herein, and the information is used in conjunction with information obtained from detecting one or more CVDA polymorphic variants, haplotypes, and/or alleles.

In certain embodiments, at least 50%, at least 60%, at least 70%, at least 80%, at least 90%, or more (e.g., 100%) of the probes or primers in a kit are designed for the detection of a polymorphic variant, haplotype, or allele that is associated with cardiovascular disease. Thus, inventive kits are distinct from microarrays, also referred to as “chips” that contain probes capable of hybridizing to and detecting a wide variety of nucleic acids, e.g., a wide variety of SNPs. In particular, certain kits of the invention are distinct from the chips that were used to identify the SNPs disclosed herein, although such chips could be used to practice the inventive methods. Inventive kits of the invention that contain chips comprising one or more probes that detect a polymorphic variant of a SNP disclosed herein differ from such chips at least in the fact that they contain a high proportion of probes that are selected to detect SNPs and/or polymorphic variants that are associated with cardiovascular disease and/or are otherwise optimized for the detection of polymorphic variants of CVDA SNPs. In certain embodiments, at least 50%, at least 60%, at least 70%, at least 80%, at least 90%, or more (e.g., 100%) of the probes or primers in the kit are designed for the detection of a CVDA polymorphic variant, CVDA haplotype, or CVDA allele.

In certain embodiments, at least some of the probe(s), primer(s), and/or antibodies contained in a kit detect a polymorphic variant or allele that is associated with CVD, wherein the polymorphic variant or allele is described in U.S. Ser. No. 10/505,936.

Kits of the present invention can further comprise one or more additional reagents in addition to probe(s), primer(s), and/or antibodies. For example, a kit may comprise a buffer-containing solution, an enzyme such as a polymerase or ligase, nucleotides, a solution optimized for performing an enzymatic reaction using the enzyme (which may contain any necessary cofactors such as metal ions), one or more “control” nucleic acids or polypeptides, and/or a substrate (e.g., for an antibody-linked enzyme), etc. A control nucleic acid is typically a nucleic acid that has the sequence of a portion of genomic DNA or cDNA that encompasses a polymorphic region and whose sequence at the polymorphic position is known. A control polypeptide is typically a polypeptide that has the sequence of a portion of polypeptide encoded by genomic DNA or encoded by a cDNA that encompasses a polymorphic region and whose sequence at the polymorphic position is known. For example, when a kit comprises probe(s) and/or primer(s) for the detection of polymorphic variants, alleles, etc., the sequence of a positive control nucleic acid at the polymorphic position may be the same as that of a polymorphic variant that is associated with cardiovascular disease, while the sequence of a negative control nucleic acid will be the same as that of a polymorphic variant that is not associated with cardiovascular disease. In the case of a kit that contains antibodies for the detection of polymorphic proteins, a positive control polypeptide may be a polymorphic protein whose sequence at the polymorphic site is the same as that of a polymorphic variant that is associated with cardiovascular disease, while the sequence of a negative control polypeptide will be the same as that of a polymorphic variant that is not associated with cardiovascular disease.

An identifier, e.g., a bar code, radio frequency ID tag, etc., may be present in or on the kit. The identifier can be used, e.g., to uniquely identify the kit for purposes of quality control, inventory control, tracking, movement between workstations, etc.

Kits will generally include one or more vessels or containers so that certain of the individual reagents may be separately housed. The kits may also include a means for enclosing the individual containers in relatively close confinement for commercial sale, e.g., a plastic box, in which instructions, packaging materials such as styrofoam, etc., may be enclosed.

According to certain embodiments, kits are manufactured in accordance with good manufacturing practices as required for FDA-approved diagnostic kits.

Drug Targets and Screening Methods

According to certain embodiments, nucleotide sequences derived from genes disclosed herein and polypeptide sequences encoded by genes disclosed herein are useful targets to identify cardiovascular drugs, e.g., compounds that are effective in treating or preventing one or more symptoms or signs of cardiovascular disease. A “symptom” refers to a manifestation of a disease or condition that is perceived by the individual who has the condition, while a “sign” refers to a manifestation of a disease or condition that is detected by clinical diagnosis, a laboratory test, an imaging procedure, or the like. It will be appreciated that the compounds identified according to the inventive methods have a number of uses in addition to their utility for the treatment and/or prevention of cardiovascular disease. For example, such compounds can serve as lead compounds for the development of useful therapeutic agents (which term is intended to encompass agents administered for purposes of treating an existing disease or condition and agents administered prophylactically, i.e., prior to the development of a particular disease, condition, symptom, or sign). Additionally or alternatively, such compounds can also be used to gain an improved understanding of the biological functions and activities of the CVDA genes and their encoded polypeptides.

Drug targets include without limitation (i) isolated nucleic acids derived from the genes that contain the polymorphisms disclosed herein; and (ii) isolated peptides and polypeptides encoded by genes that contain the polymorphisms disclosed herein.

In certain embodiments, an isolated nucleic acid comprising one or more polymorphic positions is tested in vitro or in vivo (e.g., within intact cells) for its ability to bind test compounds in a sequence-specific manner. In certain embodiments, inventive methods comprise: (i) providing a first nucleic acid containing a particular sequence at a polymorphic position, e.g., a position of a CVDA polymorphism; (ii) contacting the nucleic acid with a test compound under conditions appropriate for binding; and (iii) identifying a compound that binds selectively to the first nucleic acid. In some embodiments, inventive methods comprise further providing a second nucleic acid whose sequence is identical to that of the first nucleic acid except that it has a different sequence at the same polymorphic position; and identifying a compound that binds selectively to one of the nucleic acids. For example, the first nucleic acid may contain a CVDA polymorphic variant and the second nucleic acid may contain a different polymorphic variant at the same position that is not associated with cardiovascular disease, and the method may identify a compound that binds selectively or preferentially to the nucleic acid that comprises the CVDA polymorphic variant. Any suitable method can be used to assay binding including direct methods such as isolating a complex containing the nucleic acid and the compound, detecting a label associated with a compound that has bound to the nucleic acid, etc. Functional assays can also be used. For example, an siRNA or shRNA that specifically binds to a nucleic acid can be identified by contacting a cell with the siRNA or expressing the shRNA in a cell and determining whether expression of an mRNA transcribed from a gene that includes a CVDA polymorphism and/or expression of a polypeptide encoded by the gene is inhibited.

Selective binding as used herein refers to any measurable difference in any parameter of binding, such as, e.g., binding affinity, binding capacity, etc. In some embodiments, an agent exhibits selective binding in that its binding affinity (as determined by Ka) towards a first nucleic acid or polypeptide under the particular conditions tested is, for example, at least 5-fold greater than its binding affinity towards at least 90% of the other nucleic acids or polypeptides that would be present in a typical cell or cell lysate. In other embodiments the Ka is at least 10, 20, 50, or 100-fold greater.

In certain embodiments, an isolated peptide or polypeptide comprising one or more polymorphic positions is tested in vitro or in vivo for its ability to bind a test compound in a sequence-specific manner. Certain of the screening methods involve (i) providing a first polypeptide containing a particular sequence at a polymorphic position, e.g., a position of a CVDA polymorphism; (ii) contacting the polypeptide with a test compound under conditions appropriate for binding; and (iii) identifying a compound that binds selectively to the first polypeptide. In some embodiments, inventive methods comprise also providing a second polypeptide whose sequence is identical to that of the first polypeptide except that it has a different sequence at the same polymorphic position; and identifying a compound that binds selectively to one of the polypeptide. For example, the first polypeptide may contain a CVDA polymorphic variant and the second nucleic acid may contain a polymorphic variant at the same position that is not associated with cardiovascular disease, and the method may identify a compound that binds selectively or preferentially to the polypeptide that comprises the CVDA polymorphic variant. Any suitable method can be used to assay binding including direct methods such as isolating a complex containing the polypeptide and the compound, detecting a label associated with a compound that has bound to the polypeptide, etc. A variety of immunological methods known in the art can be used for detecting specific binding agents. Functional assays can also be used in the case of polypeptides that have a known biological function or biochemical activity.

Agents such as antisense molecules, siRNAs, shRNAs, ribozymes, other nucleic acids, peptides or polypeptides, small molecules, etc., can be tested to determine whether they modulate the expression of a CVDA gene. In certain embodiments, the invention provides methods for identifying an agent that modulates expression of a CVDA polynucleotide or polypeptide comprising steps of: (i) providing a sample comprising cells that express a CVDA polynucleotide or polypeptide; (ii) contacting the cells with a test compound; (iii) determining whether the level of expression of the polynucleotide or polypeptide in the presence of the compound is increased or decreased relative to the level of expression or activity of the polynucleotide or polypeptide in the absence of the compound; and (iv) identifying the compound as a modulator of the CVDA polynucleotide or polypeptide if the level of expression or activity of the CVDA polynucleotide or polypeptide is higher or lower in the presence of the compound relative to its level of expression or activity in the absence of the compound.

Expression of a CVDA polynucleotide or polypeptide can be measured using a variety of methods well known in the art in order to determine whether any candidate agent increases or decreases expression (or for other purposes). In general, any measurement technique capable of determining RNA or protein presence or abundance may be used for these purposes. For RNA such techniques include, but are not limited to, microarray analysis. For information relating to microarrays and also RNA amplification and labeling techniques, which may also be used in conjunction with other methods for RNA detection, see, e.g., Lipshutz, R., et al., Nat Genet., 21(1 Suppl):20-4, 1999; Kricka L., Ann. Clin. Biochem., 39(2), pp. 114-129; Schweitzer, B. and Kingsmore, S., Curr Opin Biotechnol February 2001; 12(1):21-7; Vineet, G., et al., Nucleic Acids Research, 2003, Vol. 31, No. 4.; Cheung, V., et al., Nature Genetics Supplement, 21:15-19, 1999; Methods Enzymol, 303:179-205, 1999; Methods Enzymol, 306: 3-18, 1999; M. Schena (ed.), DNA Microarrays: A Practical Approach, Oxford University Press, Oxford, UK, 1999. See also U.S. Pat Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,695; 5,624,711; 5,639,603; 5,658,734; 6,235,483; WO 93/17126; WO 95/11995; WO 95/35505; EP 742 287; EP 799 897; U.S. Pat. Nos. 5,514,545; 5,545,522; 5,716,785; 5,932,451; 6,132,997; 6,235,483; US Patent Application Publication 20020110827.

Other methods for detecting expression of CVDA polynucleotides include Northern blots, RNAse protection assays, reverse transcription (RT)-PCR assays, real time RT-PCR (e.g., Taqman™ assay, Applied Biosystems), SAGE (Velculescu et al. Science, vol. 270, pp. 484-487, October 1995), Invader® technology (Third Wave Technologies), etc. See, e.g., Eis, P. S. et al., Nat. Biotechnol. 19:673, 2001; Berggren, W. T. et al., Anal. Chem. 74:1745, 2002, etc. Methods for detecting DEA polypeptides include, but are not limited to, immunoblots (Western blots), immunofluorescence, flow cytometry (e.g., using appropriate antibodies), mass spectrometry, and protein microarrays (Elia, G., Trends Biotechnol, 20(12 Suppl): S19-22, 2002, and reference therein).

The invention also provides methods for identifying an agent that modulates expression or activity of a CVDA polynucleotide or polypeptide comprising steps of: (i) providing a sample comprising a CVDA polynucleotide or polypeptide; (ii) contacting the sample with a test compound; (iii) determining whether the level of expression or activity of the polynucleotide or polypeptide in the presence of the test compound is increased or decreased relative to the level of expression or activity of the polynucleotide or polypeptide in the absence of the compound; and (iv) identifying the test compound as a modulator of the expression or activity of the CVDA polynucleotide or polypeptide if the level of expression or activity of the CVDA polynucleotide or polypeptide is higher or lower in the presence of the compound relative to its level of expression or activity in the absence of the compound. In certain embodiments, a sample comprises cells that express the CVDA polypeptide. Agents to be screened may include any of those discussed above. Agents identified according to the above methods may be further tested in subjects, e.g., humans or other animals.

In certain embodiments, a multiplicity of compounds are tested either individually or in combination for their ability to bind to a nucleic acid or polypeptide comprising a CVDA polymorphism. High-throughput screening methods can advantageously be used.

Compounds suitable for screening according to the above methods include small molecules, natural products, peptides, nucleic acids, etc. In some embodiments test compounds are screened from large libraries of synthetic or natural compounds. Numerous means are currently used for random and directed synthesis of small molecules, saccharides, peptide, and nucleic acid based compounds. Synthetic compound libraries are commercially available from, e.g., Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Additionally or alternatively, libraries of natural compounds in the form of bacterial, fungal, plant and animal extracts are available from e.g. Pan Laboratories (Bothell, Wash.) or MycoSearch (N.C.), or are readily producible. Additionally or alternatively, natural and synthetically produced libraries and compounds are readily modified through conventional chemical, physical, and biochemical means.

Another representative example of a library is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan, et al., “Stereoselective Synthesis of Over Two Million Compounds Having Structural Features Both Reminiscent of Natural Products and Compatible with Miniaturized Cell-Based Assays”, Am. Chem Soc. 120, 8565-8566, 1998; Floyd C D, Leblanc C, Whittaker M, Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. In certain embodiments, the methods are performed in a high-throughput format using techniques that are well known in the art, e.g., in multiwell plates, using robotics for sample preparation and dispensing, etc. Representative examples of various screening methods may be found, for example, in U.S. Pat. No. 5,985,829, U.S. Pat. No. 5,726,025, U.S. Pat. No. 5,972,621, and U.S. Pat. No. 6,015,692. The skilled practitioner will readily be able to modify and adapt these methods as appropriate.

Molecular modeling can be used to identify a pharmacophore for a particular target, e.g., the minimum functionality that a molecule must have to possess activity at that target. Such modeling can be based, for example, on a predicted structure for the target (e.g., a two-dimensional or three-dimensional structure). Software programs for identifying such potential lead compounds are known in the art, and once a compound exhibiting activity is identified, standard methods may be employed to refine the structure and thereby identify more effective compounds. For example computer-based screening can be used to identify small organic compounds that bind to concave surfaces (pockets) of proteins, can identify small molecule ligands for numerous proteins of interest (Huang, Z., Pharm. & Ther. 86: 201-215, 2000). In silico discovery of small molecules that bind to a protein of interest will typically involve, for example pharmacophore-aided database searches, virtual protein-ligand docking, and/or structure-activity modeling. For example, the computer program DOCK and variants thereof is widely used (Lorber, D. and Shoichet, B., Protein Science, 7:938-950, 1998). Other non-limiting examples of suitable programs include Autodock and Flexx. It is noted that such programs and the hardware used to run them have undergone significant improvement since their introduction. Databases providing compound structures suitable for virtual screening are available in the art. For example, ZINC is a database that provides a library of 727,842 molecules, each with 3D structure, which was prepared using catalogs of compounds that are commercially available (Irwin J J and Shoichet B K. J Chem Inf Model., 45(1):177-82, 2005). Each molecule in the library contains vendor and purchasing information and is ready for docking using a number of popular docking programs. In some embodiments, the structure of a CVDA polypeptide is screened against a database using a computer-based method to identify small molecules that bind to the polypeptide. Assays to identify and/or to confirm molecules that bind to a polypeptide could include functional assays, e.g., assessing the ability of a compound to prevent blood coagulation. Radioligand binding assays, competition assays, immunologically based assays, etc., may also be used.

Intact cells or whole animals, e.g., transgenic non-human animals expressing polymorphic variants of genes disclosed herein can be used in screening methods to identify candidate cardiovascular drugs.

In certain embodiments, a cell line is established from an individual exhibiting a particular genotype with respect to one or more CVDA polymorphisms. In certain embodiments, cells (including without limitation mammalian, insect, yeast, or bacterial cells) are programmed to express a gene comprising one or more polymorphic sequences by introduction of appropriate DNA. Identification of candidate compounds can be achieved using any suitable assay, including without limitation (i) assays that measure selective binding of test compounds to particular polymorphic variants of proteins encoded by genes disclosed herein; (ii) assays that measure the ability of a test compound to modify (e.g., inhibit or enhance) a measurable activity or function of proteins encoded by genes disclosed herein; and (iii) assays that measure the ability of a compound to modify (e.g., inhibit or enhance) the transcriptional activity of sequences derived from the promoter or other regulatory regions of genes disclosed herein.

In general, a screen for a ligand that specifically binds to any particular polypeptide may comprise steps of contacting the polypeptide with a candidate ligand under conditions in which binding can take place; and determining whether binding has occurred. Any appropriate method for detecting binding, many of which are known in the art, may be used. One of ordinary skill in the art will be able to select an appropriate method taking into consideration, for example, whether the candidate ligand is a small molecule, peptide, nucleic acid, etc. For example, the candidate ligand may be tagged, e.g., with a radioactive molecule, fluorescent molecule, etc. The polypeptide can then be isolated, e.g., immunoprecipitated from the container in which the contacting has taken place (for methods performed entirely in vitro) or from a cell lysate, and assayed to determine whether radiolabel has been bound. This approach may be particularly appropriate for small molecules. Binding can be confirmed by any of a number of methods, e.g., radiolabel assays, plasmon resonance assays, etc. Phage display represents another method for the identification of ligands that specifically bind to polypeptides. In addition, determination of the partial or complete three-dimensional structure of a polypeptide (e.g., using nuclear magnetic resonance, X-ray crystallography, etc.) may facilitate the design of appropriate ligands.

Functional assays may also be used to identify ligands, particularly ligands that behave as agonists or antagonists, activators, or inhibitors of particular polypeptides. For example, a polypeptide of interest may possess a measurable or detectable functional activity and that functional activity may be increased or decreased upon binding of the ligand. Non-limiting examples of functional activities of a polypeptide include the ability to catalyze a chemical reaction either in vitro or in a cell, and/or the ability to induce a change of any sort in a biological system, e.g., a change in cellular phenotype, a change in gene transcription, a change in membrane current, a change in intracellular or extracellular pH, a change in the intracellular or extracellular concentration of an ion, etc. when present within a cell or when applied to a cell.

Thus, in certain embodiments, the invention provides methods for screening for a ligand for a CVDA polypeptide comprising steps of: (i) providing a sample comprising a CVDA polypeptide; (ii) contacting the sample with a candidate compound; (iii) determining whether the level of activity of the polypeptide in the presence of the compound is increased or decreased relative to the level of activity of the CVDA polypeptide in the absence of the compound; and (iv) identifying the compound as a ligand of the CVDA polypeptide if the level of activity of the CVDA polypeptide is higher or lower in the presence of the compound relative to its level of activity in the absence of the compound. In certain embodiments of the method the sample comprises cells that express the CVDA polypeptide. In some embodiments the CVDA polypeptide is encoded by a gene that comprises a polymorphic variant associated with cardiovascular disease.

In certain embodiments, transgenic non-human animals (e.g., rodents such as mice or rats) are created in which (i) one or more human genes disclosed herein, having different sequences at particular polymorphic positions are stably inserted into the genome of the transgenic animal; and/or (ii) the endogenous animal counterparts of genes disclosed herein are inactivated and replaced with human genes disclosed herein, having different sequences at particular polymorphic positions. Such transgenic non-human animals are encompassed within the scope of the invention. See, e.g., Coffman, Semin. Nephrol. 17:404, 1997; Esther et al., Lab. Invest. 74:953, 1996; Murakami et al., Blood Press. Suppl. 2:36, 1996. Such animals can be treated with candidate compounds and monitored for one or more indicators of cardiovascular disease. Any indicator can be assessed, and a wide variety of methods can be employed. For example, imaging, measurement of lipid levels, histopathologic examination, mortality rate, etc., can be used as an indicator.

In certain embodiments a candidate compound, e.g., a compound identified according to any of the methods described above, is administered to an animal model of cardiovascular disease, and the effect of the compound on the development of cardiovascular disease in the animal, is monitored. For example, any of the screening methods can include a step of administering the compound to an animal suffering from or at risk of developing a cardiovascular disease and evaluating the response. Response can be evaluated in any of a variety of ways, e.g., by assessing clinical features, laboratory data, blood vessel images, etc. A comparison may be performed with similar animals who did not receive the compound or who received a lower or higher amount of the compound. A number of animal models (e.g., mouse, rat, rabbit, pig, etc.) for cardiovascular diseases are known in the art. Such models may involve genetic alterations, administration of drugs, etc., to induce the development of a cardiovascular disease.

Certain embodiments provide transgenic non-human animals (e.g.,. mammals such as mice or rats) in which the endogenous counterpart of a human gene disclosed herein is “knocked out” or mutated. Such animals may, without limitation, serve as useful animal models for cardiovascular disease and may be used for testing candidate compounds.

Methods of Treatment and Pharmaceutical Compositions

As mentioned above, certain of the CVDA polymorphisms may play a causative role in cardiovascular disease. Certain embodiments provide methods of treating or preventing a cardiovascular disease comprising administering an agent that modulates the expression or activity of a CVDA gene or expression product thereof to an individual in need of treatment or prevention of cardiovascular disease. The agent can be any of the CVDA nucleic acids, polypeptides, antibodies, or ligands described above. In an exemplary embodiment, if a CVDA polymorphic variant is expressed at lower levels than a polymorphic variant that is not associated with cardiovascular disease, an agent that increases its expression or functional activity, or substitutes for its functional activity, can be administered. For example, the normal form of the polypeptide can be administered. In another exemplary embodiment, if activity of the CVDA polymorphic variant contributes to cardiovascular disease, an inhibitor such as an siRNA, inhibitory ligand, antibody, etc., can be administered.

Any of the agents described herein can be formulated and administered according to methods known in the art. In certain embodiments, the invention provides compositions comprising the inventive agents, e.g., compositions comprising a pharmaceutically acceptable carrier, diluent, excipient, etc. Suitable preparations, e.g., substantially pure preparations of the compounds may be combined with pharmaceutically acceptable carriers, diluents, solvents, etc., to produce an appropriate pharmaceutical composition. In certain embodiments, the invention therefore provides a variety of pharmaceutically acceptable compositions for administration to a subject comprising (i) an agent that modulates the expression or activity of a CVDA; and (ii) a pharmaceutically acceptable carrier, adjuvant, or vehicle. Inventive pharmaceutical compositions, when administered to a subject, may be advantageously administered for a time and in an amount sufficient to treat or prevent the disease or condition for whose treatment or prevention they are administered, e.g., a cardiovascular disease or condition, or a symptom of such.

In various embodiments, an effective amount of the pharmaceutical composition is administered to a subject by any suitable route of administration including, but not limited to, intravenous, intramuscular, by inhalation, by catheter, intraocularly, orally, rectally, intradermally, by application to the skin, etc.

Inventive compositions may be formulated for delivery by any available route including, but not limited to parenteral, oral, by inhalation to the lungs, nasal, bronchial, opthalmic, transdermal (topical), transmucosal, rectal, and vaginal routes. The term “parenteral” as used herein includes subcutaneous, intravenous, intramuscular, intra-articular, intra-synovial, intrasternal, intrathecal, intrahepatic, intralesional and intracranial injection or infusion techniques. In certain embodiments, such compositions are administered either orally or intravenously.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Solvents, dispersion media, coatings, antibacterial and antifungal agents, isotonic and absorption delaying agents, and the like, compatible with pharmaceutical administration may be included. Supplementary active compounds, e.g., compounds independently active against the disease or clinical condition to be treated, or compounds that enhance activity of a compound, can also be incorporated into the compositions.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining certain inventive compounds and their pharmaceutically acceptable acid addition salts.

Salts derived from appropriate bases include alkali metal (e.g., sodium and potassium), alkaline earth metal (e.g., magnesium), ammonium and N+(C1-4 alkyl)4 salts. This invention also envisions the quaternization of any basic nitrogen-containing groups of the compounds disclosed herein. Water or oil-soluble or dispersible products may be obtained by such quaternization.

A pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include, but are not limited to, physiological saline, bacteriostatic water, Cremophor EL™ (BASF, Parsippany, N.J.), phosphate buffered saline (PBS), or Ringer's solution.

Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. Such oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In all cases, the composition should be sterile, if possible, and should be fluid to the extent that easy syringability exists.

Certain pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be advantageous to include isotonic agents, for example, sugars, polyalcohols such as manitol, sorbitol, and/or sodium chloride in the composition. Prolonged absorption of injectable compositions can be brought about by including in the composition an agent which delays absorption, for example, aluminum monostearate and gelatin. Prolonged absorption of oral compositions can be achieved by various means including, but not limited to, encapsulation.

Sterile injectable solutions can be prepared by incorporating the active compound in the required amount in an appropriate solvent with one or a combination of ingredients enumerated above, as required, followed by filtered sterilization. In certain embodiments, solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, certain methods of preparation are vacuum drying and freeze-drying which yields a powder of the active ingredient plus any additional desired ingredient from a previously sterile-filtered solution thereof

Oral compositions generally include an inert diluent or an edible carrier. For the purpose of oral therapeutic administration, the active compound can be incorporated with excipients and used in the form of tablets, troches, or capsules, e.g. gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive compositions are advantageously delivered in the form of an aerosol spray from a pressured container or dispenser which contains a suitable propellant, e.g., a gas such as carbon dioxide, or a nebulizer. Liquid or dry aerosol (e.g., dry powders, large porous particles, etc.) can be used. The present invention also contemplates delivery of compositions using a nasal spray.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2_octyldodecanol, benzyl alcohol and water.

For local delivery to the eye, the pharmaceutically acceptable compositions may be formulated as micronized suspensions in isotonic, pH adjusted sterile saline, or, in certain embodiments, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of the present invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Systemic administration can also be by transmucosal or transdermal means. For transmucosal or transdermal administration, penetrants appropriate to the barrier to be permeated may be in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Compounds can also be prepared in the form of suppositories (e.g., with conventional suppository bases such as cocoa butter and other glycerides) or retention enemas for rectal delivery.

In certain embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polyethers, and polylactic acid. Methods for preparation of such formulations will be apparent to those skilled in the art. Certain of the materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811 and other references listed herein. Liposomes, including targeted liposomes (e.g., antibody targeted liposomes) and pegylated liposomes have been described (Hansen C B, et al., Biochim Biophys Acta. 1239(2):133-44,1995; Torchilin V P, et al., Biochim Biophys Acta, 1511(2):397-411, 2001; Ishida T, et al., FEBS Lett. 460(1):129-33, 1999). One of ordinary skill in the art will appreciate that the materials and methods selected for preparation of a controlled release formulation, implant, etc., should be such as to retain activity of the compound. For example, it may be desirable to avoid excessive heating of polypeptides, which could lead to denaturation and loss of activity.

It is typically advantageous to formulate oral or parenteral compositions in unit dosage form for ease of administration and uniformity of dosage. Unit dosage form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are advantageous. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies advantageously within a range of circulating concentrations that include the ED₅₀ with little or no toxicity. The dosage can vary within this range depending upon the dosage form employed and the route of administration utilized. For any compound used in accordance with inventive methods disclosed herein, a therapeutically effective dose may be estimated initially from cell culture assays. A dose can be formulated in animal models to achieve a circulating plasma concentration range that includes the ED₅₀ as determined in cell culture. Such information can be used to more accurately determine useful doses in humans. Levels in plasma can be measured, for example, by high performance liquid chromatography.

A therapeutically effective amount of a pharmaceutical composition typically ranges from about 0.001 to 100 mg/kg body weight, about 0.01 to 25 mg/kg body weight, about 0.1 to 20 mg/kg body weight, or about 1 to 10 mg/kg, 2 to 9 mg/kg, 3 to 8 mg/kg, 4 to 7 mg/kg, or 5 to 6 mg/kg body weight, although it will be recognized that therapeutically effective amounts depend on the particular pharmaceutical composition. As such, these ranges are merely exemplary in nature. The pharmaceutical composition can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an inventive composition can include a single treatment or, in many cases, can include a series of treatments. It will be appreciated that a range of different dosage combinations can be used.

Exemplary doses include milligram or microgram amounts of the inventive compounds per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) For local administration (e.g., intranasal), doses much smaller than these may be used. It will furthermore be understood that appropriate doses depend upon the potency of the agent, and may optionally be tailored to the particular recipient, for example, through administration of increasing doses until a preselected desired response is achieved. It is understood that the specific dose level for any particular subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

The invention further provides pharmaceutical compositions comprising an agent and, optionally, one or more additional agents. The invention further provides pharmaceutical compositions comprising a plurality of agents and, optionally, one or more additional active agent(s). Such additional active agent(s) may include an agent that has a different mechanism of action to that of the first agent. In some embodiments, such an additional active agent is a statin. In some embodiments, such an additional active agent is an anti-inflammatory agent or an anti-platelet agent.

### EXAMPLES

### Example 1

### Identification of SNPs and Haplotypes Associated with Cardiovascular Disease

Materials and Methods

A group of patients with a previous history of MI were recruited through the primary hospitals and care centers from the state of North Rhine Westphalia (NRW), Germany. The study sample of MI patients was compared with age- and sex-matched control individuals recruited in the Prospective Cardiovascular Münster (PROCAM) study for the identification and validation of genetic risk factors associated with coronary artery disease and to estimate additional genetic risk. The PROCAM study is a large, ongoing prospective epidemiological study in which a cohort of more than 43,000 men and women were initially examined and then followed for the occurrence of major coronary events (defined as the occurrence of sudden cardiac death or a definite fatal or nonfatal myocardial infarction) (Assmann, G., et al., Eur. Heart. J., 19:A2-A11; Cullen, P., et al., Circulation, 96:2128, 1997; Assmann, G., et al., Circulation, 105:310, 2002). This MI incidence cohort was used to establish a set of genetic markers where significant differences in allele frequency exist between individuals having previously suffered an MI (cases) and age- and sex-matched controls having a similar risk profile based on classical risk factors.

The MI study sample was selected to be ‘enriched’ for individuals having a genetic component to their development of CVD by including only males aged<55 years, which is a clear indication of a genetic contribution to the disease phenotype based on epidemiological studies. By selecting the study sample in this manner, we increased our statistical power to detect a statistically significant association even at a smaller sample size.

In addition, all classical risk factors were recorded and used to calculate the global risk for each study sample. Global risk was calculated by the PROCAM algorithm, taking into account 8 classical risk factors,: 5 continuous variables (age, LDL cholesterol, HDL cholesterol, triglyceride level, and systolic blood pressure) and 3 discrete variables (smoking status, diabetes, and MI in family history) (described in Assmann, G., et al., Circulation, 105:310, 2002). The global risk, as well as the distribution of the classical risk factors, was used to select our healthy control sample. The controls were selected from our PROCAM study sample, which provides us with a unique control study sample, from which we can select ‘real’ control individuals that are monitored for conventional risk factors and from which we can match the control population to the cases based on global risk as well as based on age and gender. Global risk for each MI patient first and then identified a pool of PROCAM individuals that matched the global risk score. From these individuals (as we had more controls than needed) we selected those who matched, based on including the individual parameters of the PROCAM risk factors, e.g. hypertension or diabetes. The cases were scored ‘retrospectively’ based on the information our clinicians received at the time of MI. The blood samples for the lab parameters were collected within the first 24 hours after the first ‘symptoms’ so that they were reflecting the state before the MI incidence.

Without wishing to be bound by any theory, our approach to selecting cases and controls should reduce the risk of confounding factors that may bias the analysis, as cases and controls are matched according to their classical risk factors, thus allowing identification of MI-susceptibility genes and variants that are predictive of CAD and/or MI. This method of selecting cases and controls for an association study to identify polymorphisms associated with CVD is an aspect of the invention.

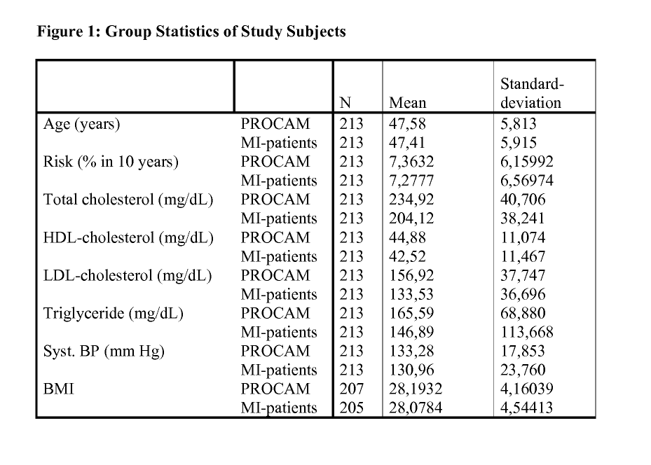

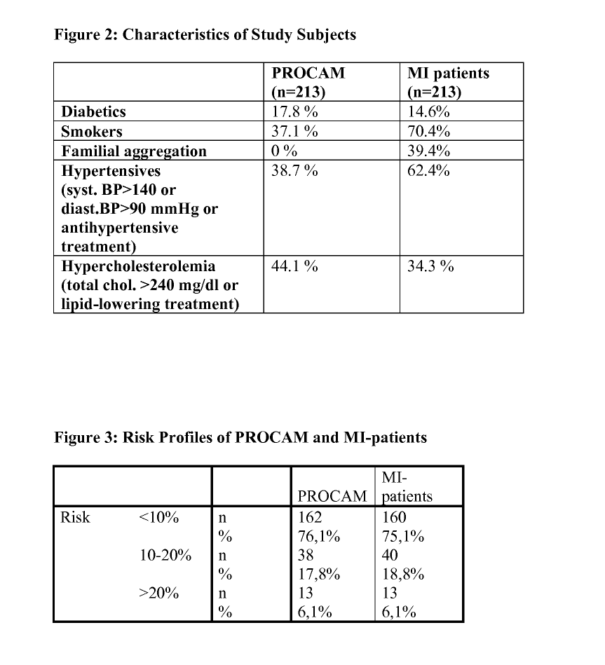

FIGS. 1-3 summarize certain characteristics of the individuals who were studied. It should be mentioned that the results described herein are based on an analysis that does not include all the individuals who participated in the study. However, it is believed that the overall characteristics of the subsets of individuals studied to date are representative of the complete groups. In FIG. 3, the Risk categories of <10%, 10-20%, and >20% refer to risk of having an acute coronary event within 10 years, as described in Assmann, supra. Individuals were classified as having a risk of <10%, 10-20%, or >20%. It will be appreciated that other methods of classifying could have been employed.

Genomic DNA was extracted from blood samples obtained from cases and controls after obtaining informed consent. The Affymetrix 500K Mendel array (Early Access) comprising two chips each allowing identification of 250,000 SNPs, was used according to the manufacturer's directions to identify a set of SNPs associated with MI (see information available at the web site having the URL www.affymetrix.com/products/arrays/specific/100k.affx, which provides information about the 50K chips that comprise the 100,000 SNP set). Briefly, the procedure was performed as follows, with minor modifications for the 500K array versus the 50K array:

a) Preparation of genomic DNA. The assay requires 250 ng of genomic DNA extracted from any biological sample. DNA is diluted into working stocks of 50 ng/uL using reduced EDTA TE buffer.

b) Restriction Enzyme Digestion. The strategy is to reduce the complexity of the genomic DNA up to 10 fold by performing a digestion with a single restriction enzyme. In order to achieve the complete coverage, two different enzymes (XbaI and HindIII) are utilized.

c) Adaptor Ligation. After digestion, the fragments are then ligated with a common set of adaptors (Adaptor NspI and StyI). These ligation adaptors recognize the cohesive four base pair overhangs. Regardless of size, all of the digested fragments are substrates for ligation.

d) PCR Amplification and Quantification. Following ligation, the sample is diluted and the complexity of the genome is further reduced via single primer PCR amplification. This generic primer recognizes the adaptor ligated fragments and is optimized to select for product sizes ranging between 250-2000 base pairs. Each sample is set up in quadruplicate and once amplification is complete, the products are combined into a single well and purified on the QIAGEN MinElute™ 96 UF PCR Purification plate. Samples are resuspended in elution buffer. The yield of the purified product is determined spectrophotometrically.

e) Fragmentation. All samples are adjusted to the same concentration for the fragmentation step. A total of 40 μg of PCR product is needed for fragmentation. PCR targets are fragmented with DNaseI, which promotes a random distribution of fragments, and are cut into millions of short pieces of <200 base pairs.

f) End-Labelling. Each fragment is then labelled with biotin. The reaction is also catalyzed with Terminal Deoxynucleotidyl Tranferase (TDT) as a combination of both achieves the most efficient labelling process.

g) Hybridization. A cocktail mix is prepared with the end-labelled products. This mix contains an oligonucleotide control reagent, two blocking agents (Human Cot-1 and Herring Sperm DNA), Tetramethyl Ammonium Chloride (TMAC) for increasing the melting temperatures of T-A bonds and DMSO which decreased the melting temperatures of G-C bonds. The samples are transferred onto the array and washed over it for 16 to 18 hours.

h) Washing, Staining and Scanning. The next day the Affymetrix Fluidics Station 450 is used to rinse the array disposing of any non-bound DNA products which is followed by staining. This is a three step process consisting of a Streptavidin Phycoerythin (SAPE) stain, followed by an antibody amplification step and a final stain with SAPE. Once completed, each array is scanned using the Scanner 3000.

Data Analysis

Classical association analyses for individual polymorphisms and common haplotypes was performed after genotyping all individuals. For example, analysis described herein may be based on up to 210 cases and 210 controls. To compare allele frequencies between groups we applied the Chi-Square test (2-tailed) and to compare genotype frequencies the Armitage-Trend-Test or Fisher's-Exact-Test.

Hardy-Weinberg equilibrium and linkage disequilibrium was examined for each polymorphism. Populations that are randomly mating with respect to a polymorphism with alleles 1 and 2, whose frequencies are given by p and q, respectively (where p+q=1), are expected to have genotypic proportions given by Hardy-Weinberg expectations p², 2pq, and q², for 1/1 homozygotes, 1/2 heterozygotes, and 2/2 homozygotes, respectively. Departures from Hardy-Weinberg equilibrium (HWE) can be tested by contrasting the observed genotypic distribution with that expected under HWE. Observed departures might reflect unrecognized population admixture, non-random mating for the polymorphism being examined (or variation in linkage disequilibrium with that polymorphism), genotyping error, or simply chance. We note that since the data collected for these studies was effectively a random sample of individuals who met our criteria for “cases” and “controls”, there is no expectation for departure for HWE.

We tested each polymorphism for departures from HWE and assessed the linkage disequilibrium patterns among polymorphisms. Specifically, we calculated disequilibrium coefficients (D′ and r²) for all pairs of polymorphisms and used established approaches for determining the local haplotype block structures (Gabriel et al., Science 2002, 296:2225-2229) as implemented in Haploview 3.0 (Barrett J C, et al., Bioinformatics, 21(2): 263-265, 2005). Linkage disequilibrium estimates and haplotype definitions for haplotype tagging was performed on the basis of the data obtained in the 420 individuals genotyped and cross-checked with the genotyping data and haplotype patterns available through the HapMap Consortium (available at the website having the URL www.hapmap.org). Although multilocus haplotypes can be estimated accurately in unrelated individuals of the sample sizes we have been able to ascertain (for haplotypes with frequencies greater than 0.05), it may not always be possible to assign multilocus haplotypes to individuals because of the number of possible haplotype combinations consistent with an observed genotypic configuration. Thus, we conducted our analyses considering polymorphisms individually, as well as in combination, with the combinations to be considered defined by the haplotype block structures.

Tables 1 and 2 present the results of our analysis. The contents of Tables 1 and 2 are further described above.

### Equivalents and Scope of the Invention

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. Furthermore, it is to be understood that the invention encompasses all variations, combinations, and permutations in which one or more limitations, elements, clauses, descriptive terms, etc., from one or more of the listed claims is introduced into another claim. In particular, any claim that is dependent on another claim can be modified to include one or more limitations found in any other claim that is dependent on the same base claim. In addition, it is to be understood that any particular embodiment of the present invention that falls within the prior art may be explicitly excluded from the claims. Since such embodiments are deemed to be known to one of ordinary skill in the art, they may be excluded even if not set forth explicitly herein. For example, any specific polymorphism, polymorphic variant, haplotype, gene, polynucleotide, or polypeptide can be excluded from the claims.

We claim:

1. A method for determining whether an individual has or is susceptible to development or occurrence of a cardiovascular disease or event, wherein the individual is in need of such determination, the method comprising the step of:
(a) detecting a polymorphic variant of a CVDA polymorphism in the individual or detecting a polymorphic variant in strong linkage disequilibrium with a CVDA polymorphism; or
(b) detecting a haplotype comprising a polymorphic variant of the CVDA polymorphism in the individual; or
(c) detecting an allele of a gene comprising the polymorphic variant of the CVDA polymorphism in the individual.

2. The method of claim 1, wherein the polymorphic variant is associated with an increased risk that the individual has or is susceptible to development or occurrence of a cardiovascular disease or event.

3. The method of claim 1, wherein the polymorphic variant is found within a gene selected from the group consisting of: one or more of the genes listed in Table 1 and/or Table 2.

4. The method of claim 1, wherein the detecting step comprises determining which of at least two polymorphic variants exists at a polymorphic site.

5. The method of claim 1, further comprising the step of determining an absolute risk or relative risk ratio based at least in part on the identity of the polymorphic variant.

6. The method of claim 5, wherein the determination of the absolute risk or relative risk ratio is based at least in part on at least one classical risk factor.

7. The method of claim 1, further comprising detecting polymorphic variants of one or more additional CVDA polymorphisms.

8. The method of claim 7, wherein the polymorphic variants constitute a haplotype.

9. The method of claim 8, wherein the haplotype is associated with an increased risk that the individual has or is susceptible to development or occurrence of a cardiovascular disease or event.

10. The method of claim 8, further comprising the step of determining an absolute risk or relative risk ratio based at least in part on the haplotype.

11. The method of claim 10, wherein the determination of the absolute risk or relative risk ratio is based at least in part on at least one classical risk factor.

12. The method of claim 1, wherein the cardiovascular disease or event is a myocardial infarction.

13. The method of claim 1, further comprising the step of determining, based on the identity of the polymorphic variant, that the individual is at increased risk of occurrence of a cardiovascular disease or event.

14. The method of claim 13, further comprising the step of selecting a therapeutic regimen for the individual, wherein the therapeutic regimen is selected based on the increased risk.

15. An isolated polynucleotide or polypeptide encoded by a CVDA gene, wherein said CVDA gene comprises a polymorphic variant of a CVDA polymorphism.

16. A kit comprising a plurality of probes or primers selected to detect one or more polymorphic variants of a CVDA polymorphism or a polymorphism in strong linkage disequilibrium with a CVDA polymorphism, wherein at least 10% of the probes or primers are selected to detect a polymorphism associated with cardiovascular disease.

17. The kit of claim 16, comprising a plurality of allele-specific oligonucleotides.

18. The kit of claim 16, comprising a plurality of oligonucleotides that terminate adjacent to a CVDA polymorphic site.

19. The kit of claim 16, comprising probes or primers selected to detect polymorphic variants at a plurality of different polymorphic sites.

20. The kit of claim 16, comprising a plurality of probes or primers for detecting each of a plurality of polymorphic variants.

21. The kit of claim 16, comprising probes or primers selected to detect one or more haplotypes associated with cardiovascular disease, wherein at least one of said haplotypes comprises a CVDA polymorphic variant.

22. A computer-readable medium on which is stored (i) an identifier for each of a plurality of polymorphisms listed in Tables 1 and 2 or an identifier for each of a plurality of haplotypes listed in Table 1 and (ii) an indicator of the frequency with which at least one polymorphic variant of the polymorphism exists in a plurality of individuals that have experienced a major coronary event or have been diagnosed with cardiovascular disease.

23. A computer-readable medium according to claim 27, further comprising an indicator of the absolute or relative risk for the occurrence of a cardiovascular disease or event in an individual having a disease-associated polymorphic variant of each of the polymorphisms.
